(19) United States
(12) Patent Application Publication
(10) Pub. No.: US 2005/0096530 A1
(43) Pub. Date: May 5, 2005

(54) Apparatus and method for customized report viewer

(75) Inventors: Shawni L. Daw, Redmond, WA (US); James J. Boisseranc, Snohomish, WA (US)

Correspondence Address: DAVIS WRIGHT TREMAINE, LLP, 2600 CENTURY SQUARE, 1501 FOURTH AVENUE, SEATTLE, WA, 98101-1688, US

(73) Assignee: Confirma, Inc., Kirkland, WA (US)

(21) Appl. No.: 10/977,392

(22) Filed: Oct. 29, 2004

Related U.S. Application Data
(60) Provisional application No. 60/515,689, filed on Oct. 29, 2003.

(51) Int. Cl. A61B 5/05

(52) U.S. Cl. 600/408; 600/407; 382/128

(57) ABSTRACT

A system for the automatic generation of custom report viewing utilizes imaging data and computer-aided detection technology to identify cancerous tumors. A typical collection of data from a patent's scan may include hundreds of images and associated data. The custom report viewer allows one physician, such as a radiologist, to analyze the data and prepare a report. The generated report may contain images, computed measurements, classifications based on a standard (such as the ACR BI-RADS for Breast MR), and locations relative to landmarks. Different physicians, such as an oncologist or a surgeon, may have need of differing images and supporting data for their own purposes. Each physician may select, in advance, custom selection criteria that are stored in association with that physician. A report generator module uses the stored selection criteria and report filtering to extract the images and supporting data specified by the particular physician. The system allows a surgeon to alter the selection criteria and obtain further images if necessary and permits the generation of multiple selection criteria by one physician for different purposes, such as surgical planning, therapy reporting, and the like.

### BACKGROUND OF THE INVENTION

1. Field of the Invention

The present invention is directed generally to techniques for medical image analysis and diagnosis and, more particularly, to an apparatus and method for the generation of customized report viewing.

2. Description of the Related Art

Breast cancer affects millions of individuals. In addition to breast self-examination, current medical advice includes periodic mammograms, which utilize conventional X-ray technology. Other forms of imaging, such as magnetic resonance imaging (MRI), are also known in the art.

The initial data, which includes many images and may include additional laboratory test results, is evaluated by a physician. Typically, the radiologist evaluates images to determine whether a particular lesion is cancerous. The radiology must scan all of the patient reports, diagnose the patient and generate various reports. Given the large number of images, it is very time consuming for the radiologist to select different images for different reports.

Therefore, it can be appreciated that there is a significant need for techniques to allow the efficient generation of reports that provide the details required by the surgeon. The present invention provides this and other advantages as will be apparent from the following detailed description and accompanying figures.

### BRIEF DESCRIPTION OF THE SEVERAL VIEWS OF THE DRAWING(S)

FIG. 1 illustrates the generation and dissemination of medical reports in accordance with the present teachings.

FIG. 2 is a functional block diagram of a system constructed in accordance with the present teachings.

FIG. 3 is a flow chart illustrating operation of the system of FIG. 2.

FIGS. 4-9 are examples of medical images and associated data used as the basis for the generation of a customized report by the system of FIG. 2.

FIGS. 10A-10E are graphical images of a volume of interest identified as a possible tumor and related data.

FIG. 11 illustrates a pre-treatment report, including medical images with anatomical features identified and anatomical data and measurements displayed.

FIG. 12 an illustration of medical images and data with identified anatomical features and volumes of interest and data related to pre- and post-treatment measurements.

FIG. 13 is an illustration of post-treatment reports indicating trends in treatment.

FIGS. 14A-14G illustrate a sample report.

### DETAILED DESCRIPTION OF THE INVENTION

As will be discussed in further detail, the system described herein is directed to techniques for the automatic generation of custom reports from an initial set of image data. Various physicians have need for specific images and associated data. For example, the radiologist may evaluate certain images and the associate data to identify a volume of interest (VOI). Other physicians, such as a surgeon, may require a completely different set of images and associated data. Still other physicians, such as an oncologist, may have need for still another different set of images and associated data.

The types of procedures and reports needed for a patient varies depending on the state of the patient, and the physician interested in the results. There are reports needed for the initial diagnosis or screening of cancer. Once cancer has been identified, other reports are needed for staging, or determining the extent of disease. Based on the staging, patients may have surgery, in which case the surgeon may desire to view another type of report. If the patient is to undergo therapy before or after surgery, a report for monitoring the effectiveness of the therapy would be useful.

Particular imaging techniques, such as MRI, may scan a volume of tissue within a region of anatomical interest. Scan information or data corresponding to an anatomical volume under consideration may be transformed into or reconstructed as a series of planar images or image “slices.” For example, data generated during a breast MRI scan may be reconstructed as a set of 40 or more individual image slices. Any given image slice comprises an array of volume elements or voxels, where each voxel corresponds to an imaging signal intensity within an incremental volume that may be defined in accordance with x, y, and z axes. The z axis commonly corresponds to a distance increment between image slices, that is, image slice thickness.

Any given medical imaging technology may be particularly well suited for differentiating between specific types of tissues. A contrast agent administered to the patient may selectively enhance or affect the imaging properties of particular tissue types to facilitate improved tissue differentiation. For example, MRI may excel at distinguishing between various types of soft tissue, such as malignant and/or benign breast tumors or lesions that are contrast enhanced relative to healthy breast tissue in the presence of Gadolinium DPTA or another contrast agent.

Medical imaging techniques may generate or obtain imaging data corresponding to a given anatomical region at different times or sequentially through time to facilitate detection of changes within the anatomical region from one scan to another. Temporally varying or dynamic tissue dependent contrast agent uptake properties may facilitate accurate identification of particular tissue types. For example, in breast tissue, healthy or normal tissue generally exhibits different contrast agent uptake behavior over time than cancerous tissue. Moreover, malignant lesions generally exhibit different contrast agent uptake behavior than benign lesions (“Measurement and visualization of physiological parameters in contrast-enhanced breast magnetic resonance imaging,” Paul A. Armitage et al., Medical Imaging Understanding and Analysis, July 2001, University of Birmingham).

In general, at any particular time, the intensity of an imaging signal associated with any particular voxel depends upon the types of tissues within an anatomical region corresponding to the voxel; the presence or absence of a contrast agent in such tissues; and the temporal manners in which such tissues respond following contrast agent administration. In several types of breast MRI situations, normal or healthy tissue exhibits a background signal intensity in the absence of a contrast agent, while abnormal or cancerous tissue exhibits a low or reduced signal intensity relative to the background intensity. Thus, prior to contrast agent administration, abnormal tissue typically appears darker than normal tissue. In the presence of a contrast agent, lesions or certain types of abnormal tissue typically exhibit a time-dependent enhancement of imaging signal intensity relative to the background intensity.

The imaging data is processed and classified by a Computer-Aided Detection (CAD) processor. The CAD processor may detect and/or diagnose a VOI automatically or simply identify and segment certain regions in the image based on sets of rules established by the radiologist and/or surgeon. Examples of CAD processors are described, by way of example, in U.S. application Ser. No. 09/721,913, entitled CONVOLUTION FILTERING OF SIMILARITY DATA FOR VISUAL DISPLAY OF ENHANCED IMAGE, filed Nov. 24, 2000, now allowed, and U.S. application Ser. No. 09/722,063 entitled DYNAMIC THRESHHOLDING OF SEGMENTED DATA SETS AND DISPLAY OF SIMILARITY VALUES IN A SIMILARITY IMAGE, filed Nov. 24, 2000, now pending. These applications are assigned to the assignee of the present invention and are incorporated by reference in their entirety.

In the above-referenced application entitled DYNAMIC THRESHHOLDING OF SEGMENTED DATA SETS, image slices are displayed in two dimensions as picture elements (i.e., pixels) that represent volume elements (i.e., voxels). In one exemplary embodiment described in that application, a caregiver, such as a radiologist, examines the imaged data and identifies one or more regions of interest, commonly referred to as a volume of interest (VOI). Based on the radiologist's analysis, certain voxels or discreet data elements may be identified as lesions. The CAD processor utilizes a plurality of different measures of the physical characteristics of the selected discreet data elements and places them in a training set. Thereafter, other discreet data elements, representing additional voxels, are analyzed with respect to the training set to determine a similarity value. That is, the multiple physical characteristics of each discreet data element may be compared against the multiple physical characteristics of the training set and a similarity value determined based on this analysis. Those data elements having a sufficient similarity value may be displayed as a similarity image. In such an image, all discreet data elements or voxels meeting the requirement (i.e., having sufficient similarity to the training set) may be displayed. Use of this image classification allows the detection of areas that are similar to the training set, which has been identified by the radiologist as a lesion. This analysis may be extended to discreet data elements in regions other than the region surrounding the training set to identify metastasized cancer cells.

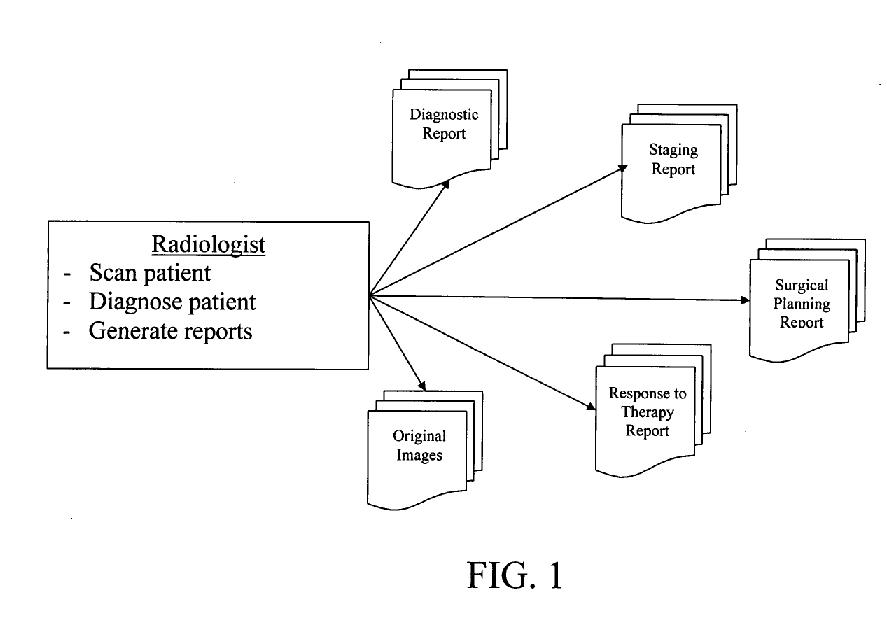

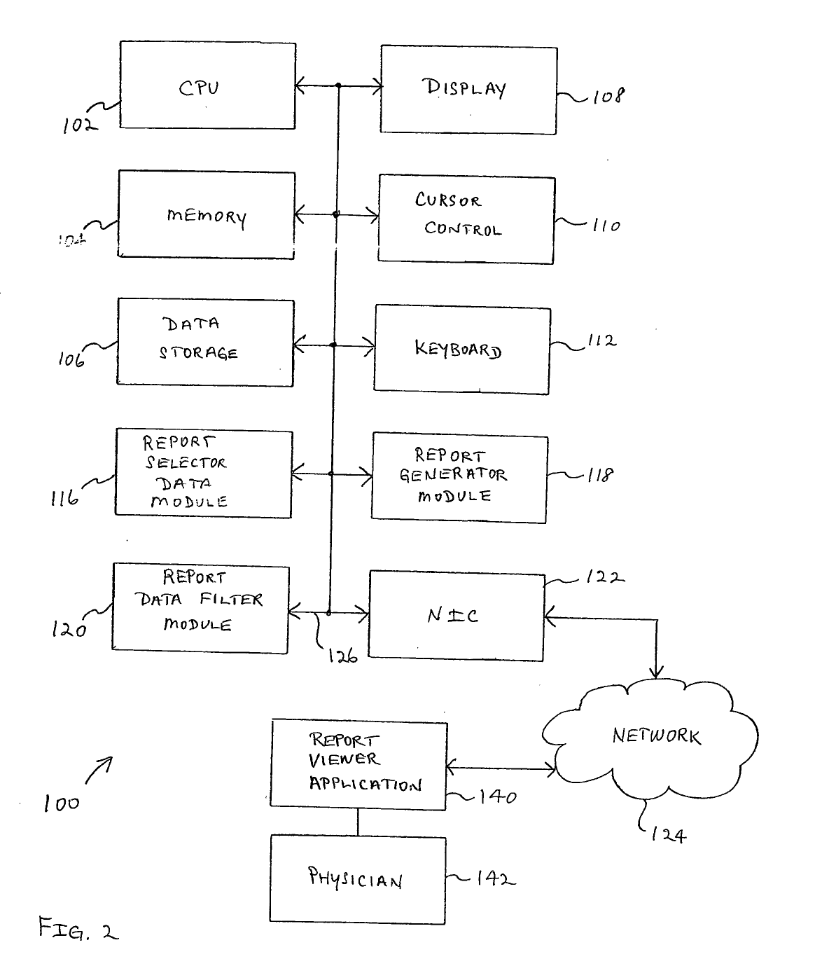

Throughout this whole process, different physicians are interested in potentially different images and sets of data. MR studies often result in thousands of images. The radiologist then is responsible for analyzing the images and identifying tissues of interest, which may vary depending on the type of report. The report may also contain information to meet the recommendations in the American College of Radiology Breast Imaging and Reporting Data System (ACR BI-RADS®) Breast Imaging Atlas. This information may be chosen by the radiologist, or automatically computed for the identified tissues of interest. FIG. 1 illustrates a number of different reports that can be created and individually customized for report types, or for different physicians or both. This feature provides a mechanism to provide custom views of imaging results for the various physicians, while minimizing the effort of the radiologist to create these reports.

Although the techniques discussed herein use examples directed to evaluation of breast tumors, the techniques are more widely applicable to the evaluation of tissue for surgical planning purposes in general.

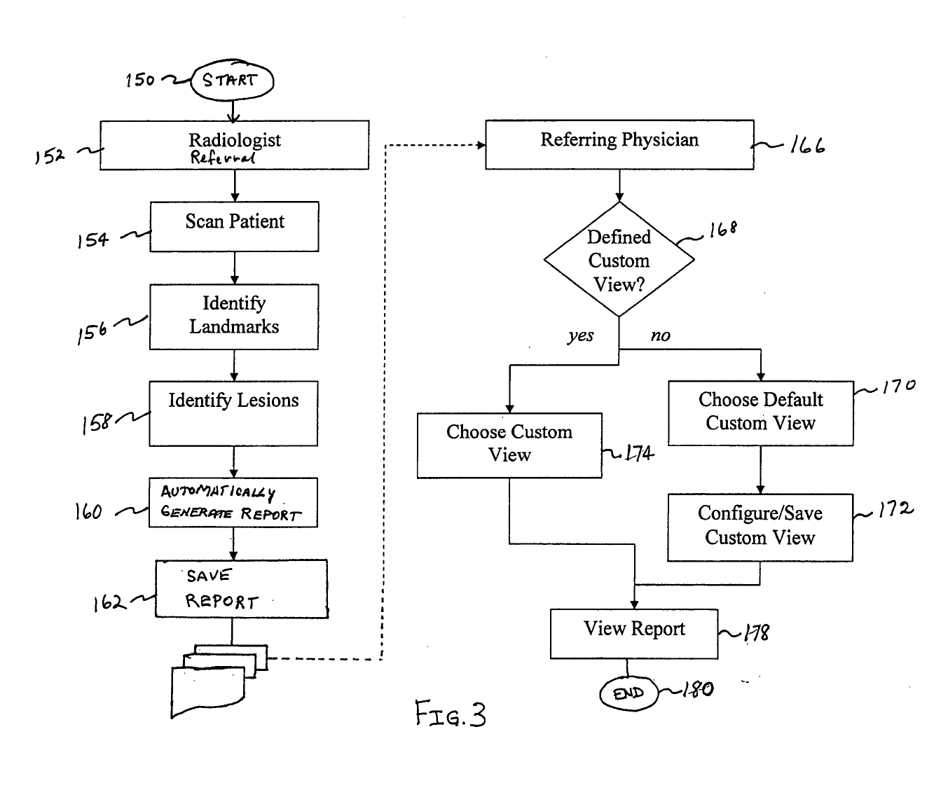

FIG. 2 is a functional block diagram of a system 100 constructed in accordance with the principles described herein. Many of the components of the system 100 are implemented as conventional computer components and need only be described briefly herein.

The system 100 includes a central processing unit (CPU) 102 and a memory 104. The CPU 102 may be implemented as a microprocessor or part of a minicomputer or mainframe computer. The CPU 102 may be a conventional microprocessor chip, microcontroller, digital signal processor, or the like. Similarly, the memory 104 may be implemented by a variety of known technologies. The memory 104 may comprise random access memory (RAM), read-only memory, flash memory, or the like, or combinations thereof. The system 100 is not limited by the specific implementation of the CPU 102 and memory 104.

The system 100 also includes data storage 106, and conventional input-output (I/O) devices, such as a display 108, cursor control device 110, and keyboard 112. The data storage 106 may be implemented in a variety of forms, such as a hard disk drive, optical drive or the like. The data storage 106 may include removable storage elements, such ad a CD, CD-R/W, DVD, DVD-R/W, or the like. The display 108 is a conventional computer display having the necessary graphic resolution to allow satisfactory display of images, as will be described below. In a typical implementation, the display 108 is a color computer display. The cursor control 110 may be a joystick, mouse, trackball or the like. The keyboard 112 may be a conventional computer keyboard or may include custom keys to simplify the processes described herein.

The data storage 106 receives and stores image data and associated data from a number of different imaging devices known in the art. Among them are conventional X-rays, computerized tomography (CT scanners), magnetic resonance imaging (MRI), positron emission tomography (PET), Single Photon-Emission Computed Tomography (SPECT), ultrasound imaging, or the like. One or more of these modalities may be used to provide imaging data to the system 100.

The system 100 also includes a report selector data module 116, a report generator module 118, and a report data filter module 120. As will be described in greater detail below, the report selector data module 116 is used by various physicians to predefine the report types and report data (e.g., images and data) that are desired by the physician. Thus, each physician may have a customized set of report selectors stored in the report selector data module 116.

The report generator module 118 utilizes the uniquely specified data in the report selector data module 116 to generate customized reports. The report data filter module 120 is used to apply the user's selector data to the patient data stored in the data storage 106. Operational details of the report selection processor provided below.

In addition to individual physicians specifying unique sets of data for reports, the report selector data module 116 can store selector data for different report types, as illustrated in FIG. 2. For example, a diagnostic report may include a first subset of patient data from the data storage 106 while a staging report contains a different subset of patient data. A surgical planning report may contain yet another different subset of patient data. These type of customized reports may be predetermined by a physician, such as the radiologist, or predetermined on the basis of an organization, such as the surgical staff, radiology staff, or the like. The system 100 advantageously provides the ability to predetermine a number of reports, which are stored in the report selector data module 116.

The system 100 also includes a network interface controller 122, which is coupled to a network 124. The network 124 may be any conventional form of network, such as a local area network (LAN), a wide area network (WAN), or the like. The network interface controller 122 may be selected based on the network type and the interface type. For example, in one embodiment, the network interface controller 122 may be an Ethernet controller. Alternatively, the network interface controller may be a USB interface, a dial-up modem or constructed in accordance with IEEE-1394 interface. The system 100 is not limited by the specific form of the network 124 nor the network interface controller 122.

The various components described above are coupled together by a bus system 126, which may include a data bus, address bus, control bus, power bus, and the like. For the sake of clarity, those various buses are illustrated in FIG. 2 as the bus system 126.

Those skilled in the art will recognize that many of the functional blocks illustrated in the functional block diagram of FIG. 2 may be implemented as standalone hardware or as a set of computer instructions stored in the memory 104 and executed by the CPU 102. For example, the report generator 118 may be implemented as a set of software instructions executed by the CPU 102. Similarly, other elements, such as the report data filter 120 may be implemented by hardware components, such as a digital signal processor, or may be implemented as a set of software instructions stored in the memory 104 and executed by the CPU 102. However, each of these blocks performs a separate function and is thus illustrated in the functional block diagram of FIG. 2 as a separate element. However, the system 100 is not limited by the specific implementation of the various components.

In a typical embodiment, the referring physician has access to the report viewer application 140. The report viewer application 140 is an application that receives the full report and displays the custom view report for the physician. The report viewer application 140 may also include an application that allows the physician to specify selection criteria for storage in the report selector data module 116. The report viewer application 140 may be included on the report CD or accessible via the network 124, or on a web page hosting the reports. The referring physician uses the report viewer application 140 to view the images and data. In this manner, the referring physician can set up custom selection criteria to identify images of interest. This may reduce 100-1,000 images to the physician's pre-selected subset of 8-10 images. Alternatively, the default configuration lists will be provided for basic report types, such as those listed in FIG. 1.

The system 100 can be readily implemented in a variety of different computer architectures. In one embodiment, the data storage 106 is a mass storage unit associated with the system 100. However, those skilled in the art will appreciate that the data storage 106 is intended to encompass not only local storage, but mass storage that may be available on the network 124, such as the LAN, or delivered to the storage area 106 at a remote location via a virtual private network (VPN) or wide area network (WAN). The location and specific form of the data storage 106 may be selected based on the particular needs of the system 100. The system 100 is not limited by the specific form of the data storage 106 nor its location with respect to the other components of the system 100.

Indeed, in a distributed model, various components of the system 100 may be remotely located from each other. For example, the imaging device (not shown) may typically be located in a radiology department of a hospital while the components of the system 100 may be located within the radiology department of a hospital or in some other location within the hospital. In yet another exemplary embodiment, the system 100 need not be within the hospital at all. The imaging data may be provided to the system 100 as a data file stored on a data storage device, or as a data file stored on a CD-ROM or transmitted over, by way of example, the network 124.

Similarly, the report generator module 118 may be located remotely from other components of the system 100. As described above, the report data filter module 120 selectively filters images and associated data using the criteria specified in the report selector data modules 116. The report generator module 118 creates a customized report for presentation to the physician.

In one embodiment, the physician may be physically present to operate the system 100. In another exemplary embodiment, the surgeon and/or radiologist may be at a computer or terminal that may be remote from the system 100. For example, the patent application entitled SYSTEM AND METHOD FOR DISTRIBUTING CENTRALLY LOCATED PRE-PROCESSED MEDICAL IMAGE DATA TO REMOTE TERMINALS, describes a system in which the CAD portion (e.g., the CAD processor) is centrally located and the physician views pre-processed data from a remote terminal. A similar architecture could be applied to the system 100 to permit the physician to view the pretreatment reports and/or post-treatment reports from a remote terminal. Distributed computing environments are well known in the art and can be readily applied to the system 100. Accordingly, the system 100 is not limited by any specific computer architecture or the requirement that the components listed in FIG. 2 be co-located.

The system 100 allows treatment of a patient to be carried out in an efficient and cost effective manner by providing a system of customized reports that are automatically generated to provide the precise data specified by physicians. Each physician may specify the criteria for one or more report types and the report generator module 118 automatically creates a variety of different report types from the same superset of images and data stored in the data storage 106. Initially a physician, typically the radiologist, must evaluate the all the images and associated data to make a diagnosis. However, the large quantity of images and associated data are cumbersome to include in a report. Furthermore, the sheer volume of such a report diminishes the usefulness of the report and increases the risk that important data may be overlooked.

Using the system 100, the radiologist may specify selection criteria in advance that indicates the type of images and data to be included in a diagnosis report and the report generator module 118 automatically selects the images and associated data specified by the radiologist in advance to create the diagnosis report. Utilizing the selection criteria, the report generator module 118 automatically generates the same type of diagnosis report for each patient. Thus, the reports have a consistency that improves quality and, at the same time, the radiologist is spared the task of manually extracting the desired images and associated data to create a report from scratch for each and every patient or for the same patient each time a new set of images is generated.

In another example, a surgeon may have need for a report, such as a surgical planning report, that requires different images and associated data than the radiologist's diagnosis report. The surgeon may specify selection criteria in advance that indicate the type of images and data to be included in the surgical planning report. The report generator module 118 applies this set of selection criteria, which may be very different from that radiologist's selection criteria for a diagnosis report, to the collection of images and data for a particular patient to automatically generate the surgical planning report. Thus, the system 100 processes the same collection of images and associated data to automatically generate two different reports that include precisely the data and images requested by the radiologist and surgeon, respectively. The selection may include not only formatting criteria, such as the sequence in which images are arranged and page layout format, but may specify certain image views, selected types of data and the like.

In another example, the surgeon may also require a different type of report to monitor pre-operative treatment of a cancerous lesion with radiation or chemotherapy. For a treatment monitoring report, the surgeon may specify in advance the set of images and data required for treatment monitoring. Again, the report generator module 118 applies the selection criteria stored in the report selector data module 116 to automatically generate the treatment monitoring report from the set of images and data already stored in the data storage 106. A treatment monitoring report may include, by way of example, the data and images previously analyzed by the radiologist that identify the detected lesions, determine measurements of lesions in three dimensions, determine measurements of the location of lesions with respect to anatomical landmarks, and the calculation of a volume of tissue for each VOI that must be removed in a surgical procedure. The treatment monitoring report may be readily stored in the data storage 106, or stored in a location remote to the system 100, such as a central storage location. In this embodiment, the pre-treatment report and associated data may be transmitted to a central storage location via the network 134 (e.g., the LAN or (WAN), in a manner well understood by those skilled in the art.

The customized report viewing feature allows radiologists to save a “full report” that a referring physician can view. The report will be a superset of all of the images desired for a report. The referring physician then customizes their view of the report. FIG. 3 is a flowchart illustrating the operation of the system 100 at a start 150, a patient may have been examined by his or her physician for further diagnosis. At step 152, a radiologist refers the patient for a diagnostic workup. In step 154, the patient is scanned. As noted above, this process may include a number of different imaging modalities. Although the images illustrated in many of the figures contained herein are MRI data, the system 100 is not limited by any specific imaging modality.

In step 156 the landmarks are identified that will be used in subsequent treatment. These landmarks may be automatically computed, or manually identified (or altered) by the radiologist. For example, breast imaging may commonly use the chest wall position, the nipple location, and the skin surface location as landmarks, wherein the position of a VOI may be indicated with respect to one or more of these landmarks.

In step 158, the radiologist identifies lesions. The identification of lesions may be done manually by the physician or with the use of a CAD processor, such as those described in the above referenced patent applications. This step may also include classifying the lesions according to some standard, such as the ACR BI-RADS. The classifications may be automatically computed, or manually specified by the radiologist.

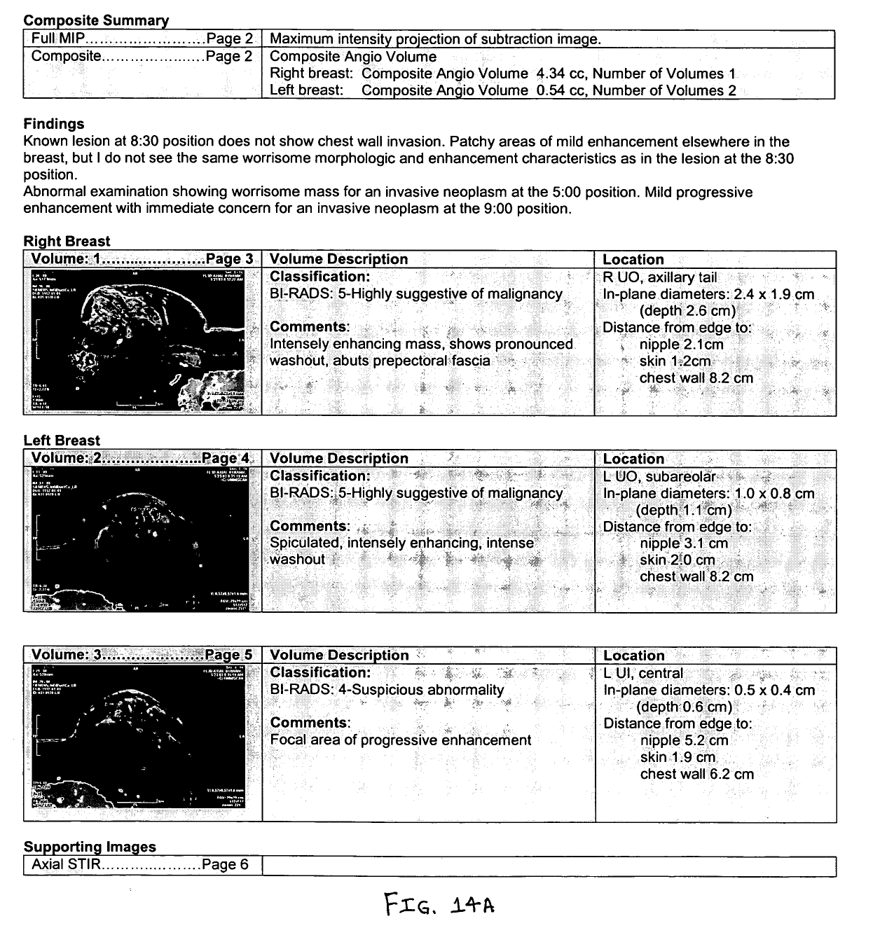

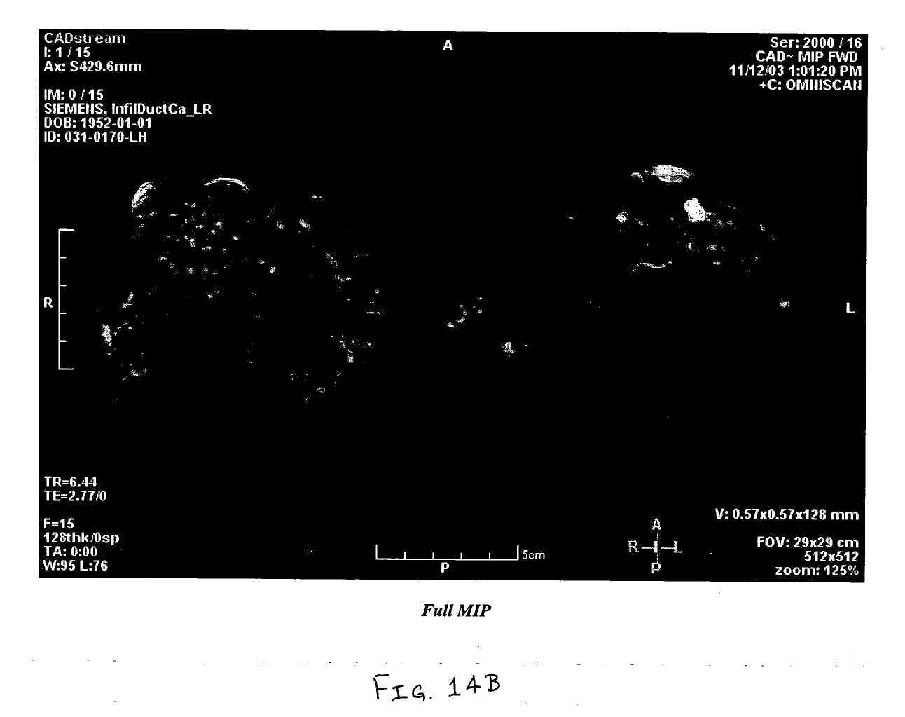

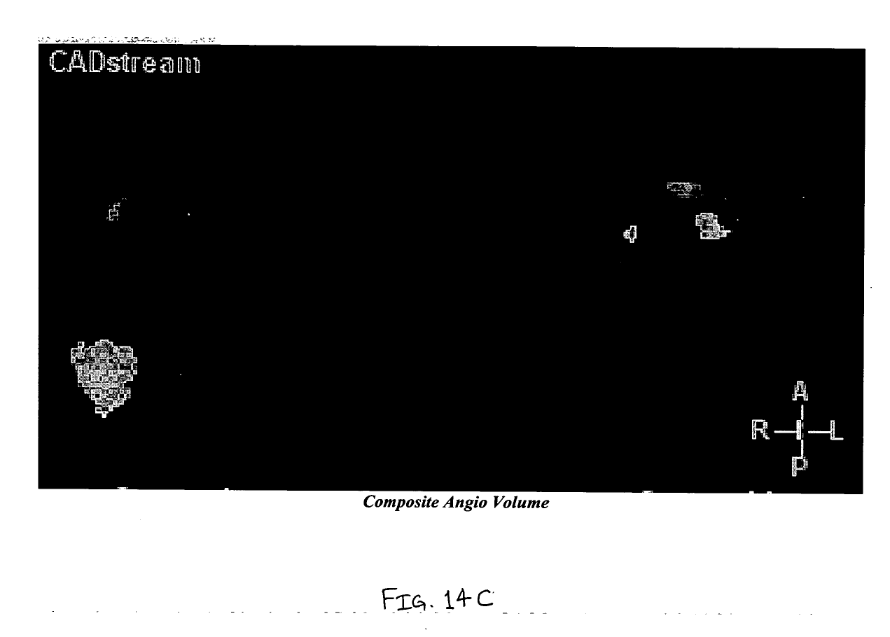

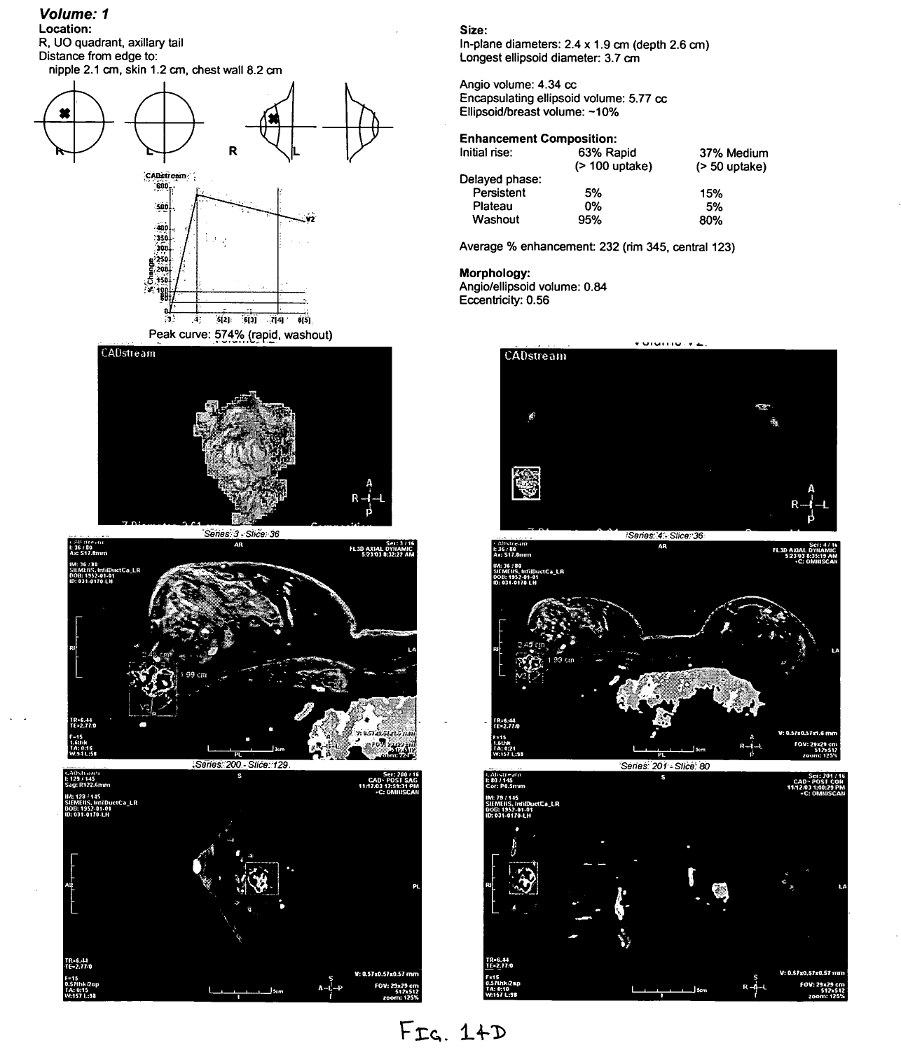

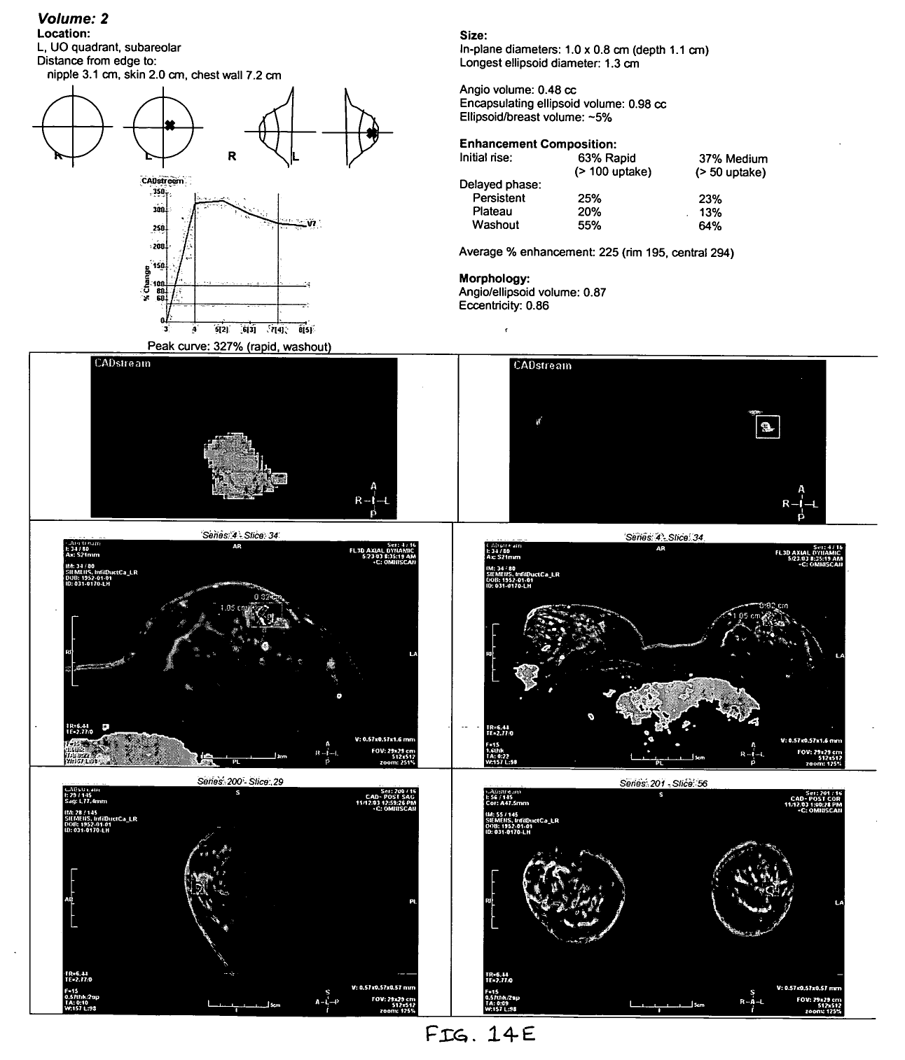

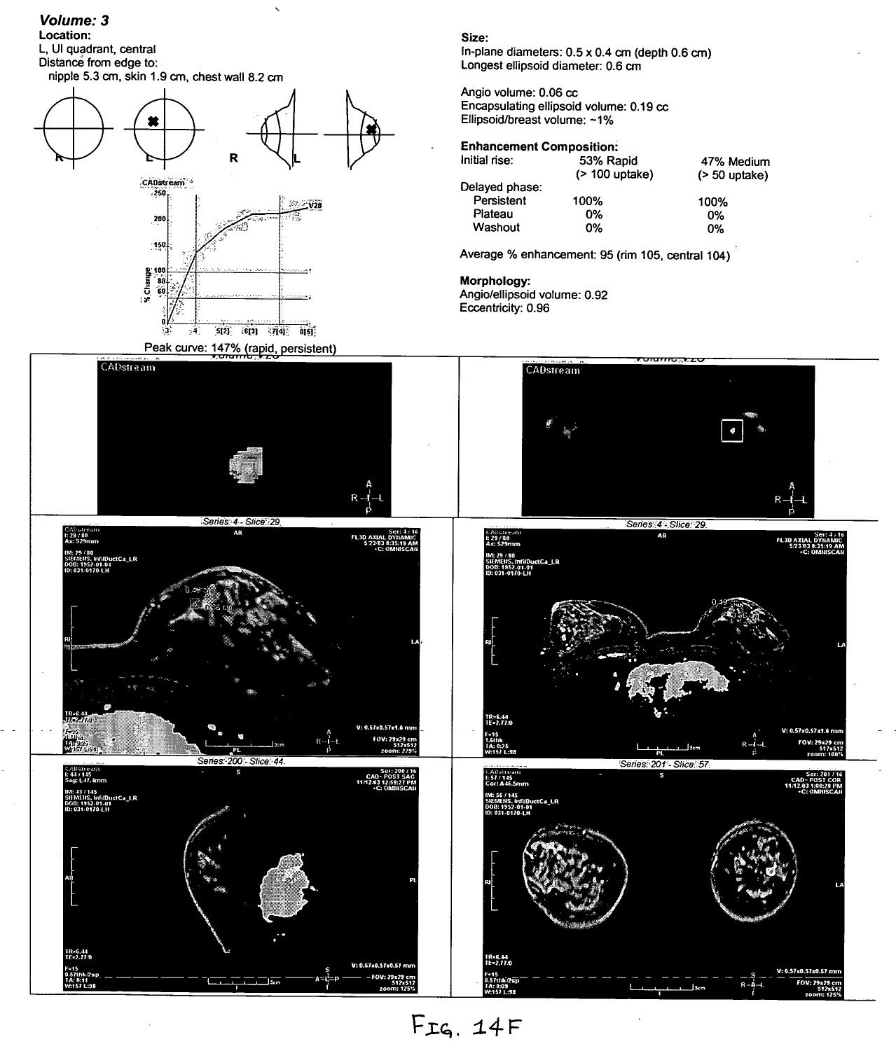

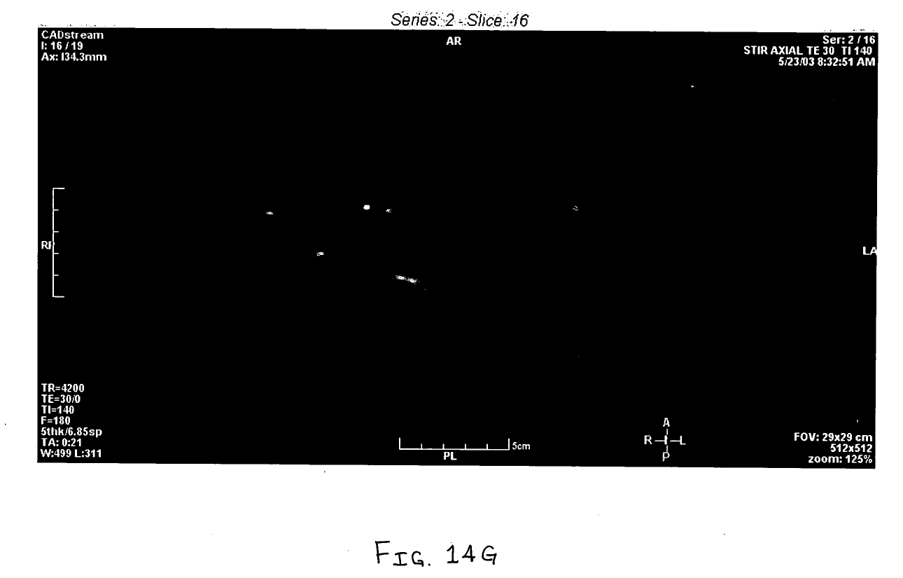

In step 160, the report is automatically generated by the CAD processor. This includes gathering all of the measurements, classifications, and images based on the lesions identified by the radiologist. FIGS. 14A-14G illustrate a sample report.

In step 162, the radiologist saves a “full report,” which will include all possible images and measurements of interest to the radiologist and referring physician, based on the landmarks and selected lesions (i.e., VOIs). The report can be saved in the data storage 106. In this embodiment, the data storage 106 may be a hard disk drive. Alternatively, the report may be stored on a CD/DVD or provided to a remote location via the network 124.

At some subsequent time after the report has been generated and stored, the referring physician will initiate a review of the report at step 166. In decision 168, the system 100 determines whether the physician has previously defined a custom view. That is, the system 100 will determine whether the particular physician has predetermined report criteria stored in the report selector data module 116. If the physician has not selected a custom view (i.e., there is no report selector criteria in the report selector data module 116 for that physician or for the type of report requested by that physician) the result of decision 168 is NO. In that event, the system 100 moves to step 170 and may select a default custom view from the report selector data module 116.

At step 172 the referring physician may configure the system to create custom views for future applications and may save the custom selection criteria in the report selector data module 116.

Returning again to decision 168, if the referring physician has defined a custom view, the result of decision 168 is “YES.” In that event, the system 100 moves to step 174 and chooses the custom view. That is, the report generator module 118 (see FIG. 2) utilizes the report selector criteria for that physician, which is stored in the report selector data module 116. The report selection criteria is used by the report data filter module 120 to selectively extract images and associated data from the data storage 106. The report generator module 118 assembles the custom selected report.

In step 178, the physician may review the report. This is either the default view report or customized default view report from steps 170-172, or the custom view report of step 174. The process ends at 180 with the physician reviewing the selected data.

Those skilled in the art will appreciate that the identification of a custom view in decision 168 occurs automatically. Thus, once a physician has generated a custom view (i.e., determined selection criteria for storage in the report selector data module 116), the system 100 automatically generates the requested type of report (e.g., a diagnosis report) for that physician in accordance with the physician's customized selection criteria. The custom selection criteria are applied to all reports of that type (e.g., a diagnosis report) so that the referring physician need not customize a report for each patient. It should be understood, however, that the physician may always alter a customized report to obtain additional data, if necessary, for a proper diagnosis or more detailed analysis.

The features of the system 100 provide a number of key advantages. The radiologist always saves the same report. The radiologist does not have to create custom reports for each type of report, or for each physician. Since the radiologist may support several physicians, this is a significant time saver for them. It also provides consistency since every patient is processed and reported the same.

In addition, the referring physician views the same report for all patients, regardless of which radiologist generated them. There is currently quite a bit of variability in the reports, depending on the radiologist who creates them. The consistency in report generation provided by the system 100 eliminates a primary source of variability.

A single report, with consistent presentation of information in a consistent format, serves the needs of the several interested physicians. For example, the radiologist is most interested in the diagnostic report, which captures the images that were important in making the diagnosis. In contrast, the surgeon is most interested in planning the surgery based on the findings, so they will likely want to see the lesions identified by the radiologist in different orientations, and relative to landmarks. In yet another example, the oncologist and/or surgeon is most interested in monitoring the response to therapy or surgery.

It should be noted that the system 100 also permits a single physician to have multiple custom views. This allows the physician to also configure the report based on the state of the patient (for example, pre-operative or post-operative), or based on the intended use of the report by the physician.

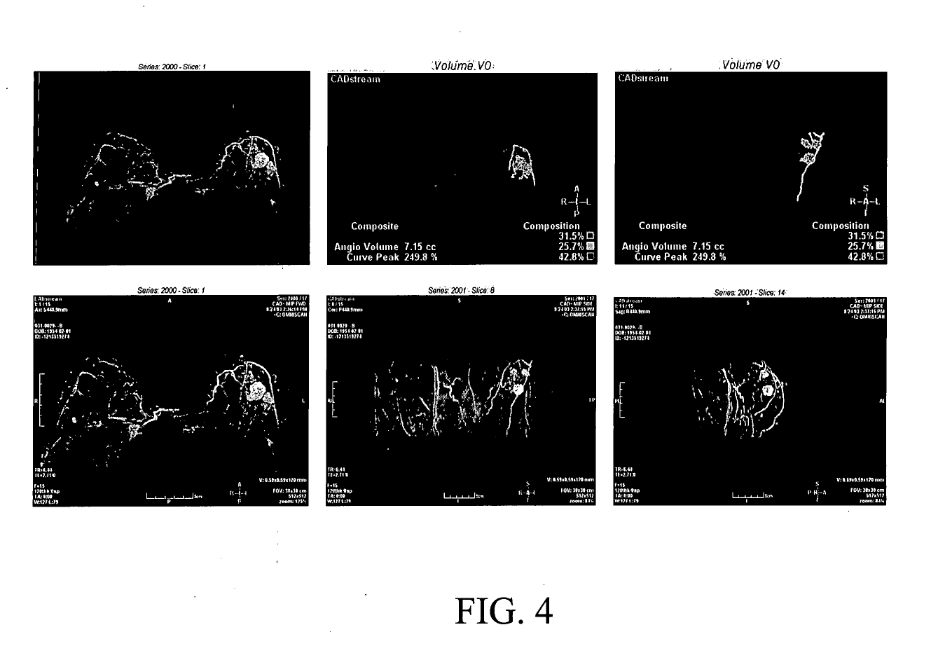

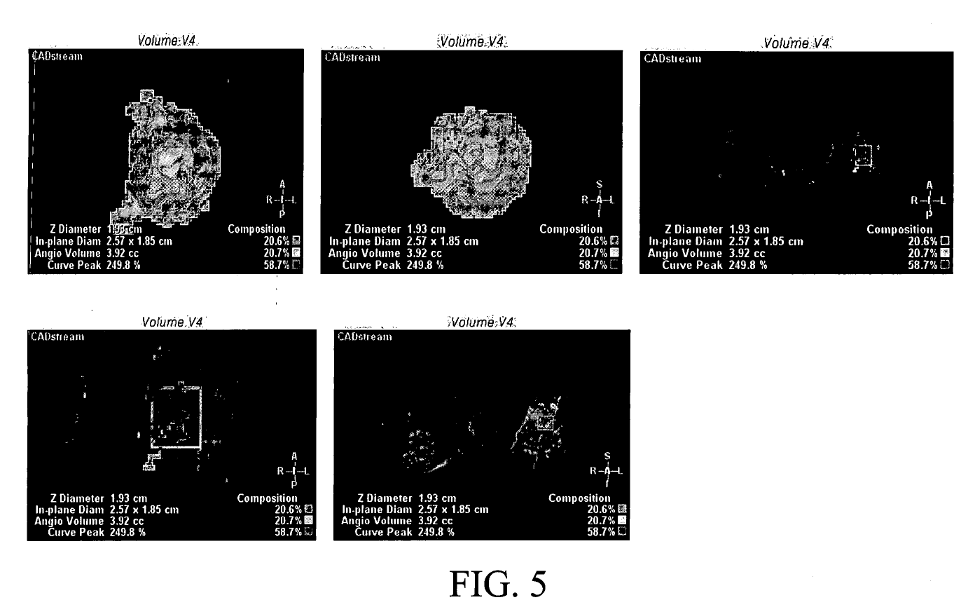

FIGS. 4-9 illustrate a sample series of images that are stored in the data storage 106 (see FIG. 2) as a complete set of images for a patient. Selected images may be extracted to form the basis for various customized reports. FIG. 4 illustrates global images of breasts and identified lesions. FIG. 5 illustrates specific examples of lesions and location within the breast. In addition, the images have measurement data relating to the size and location of the lesion.

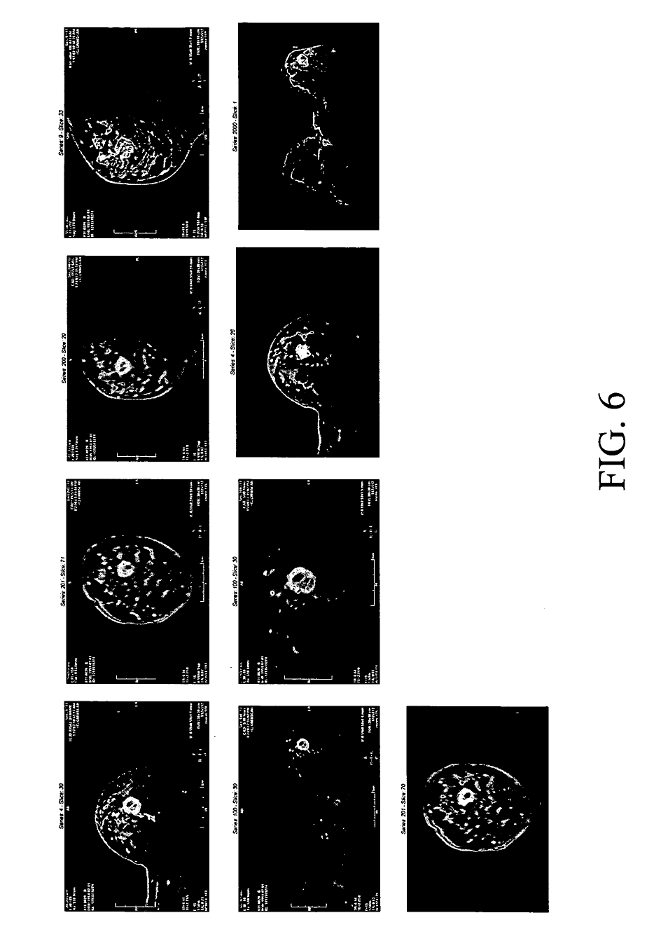

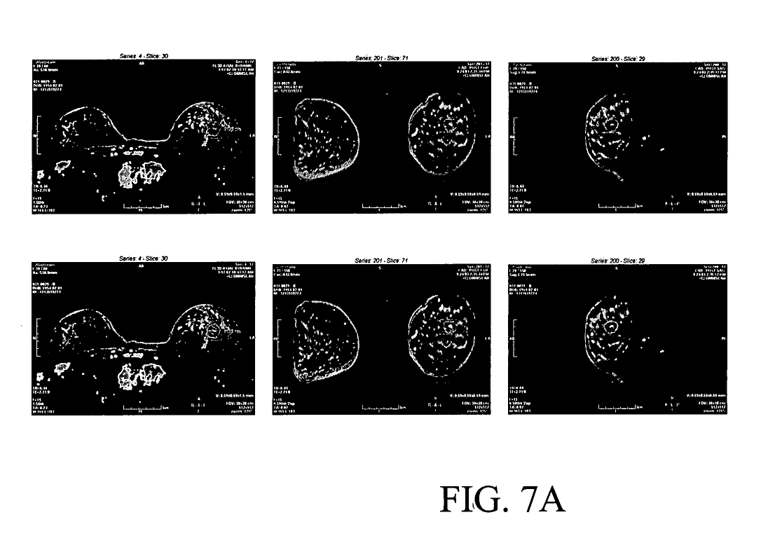

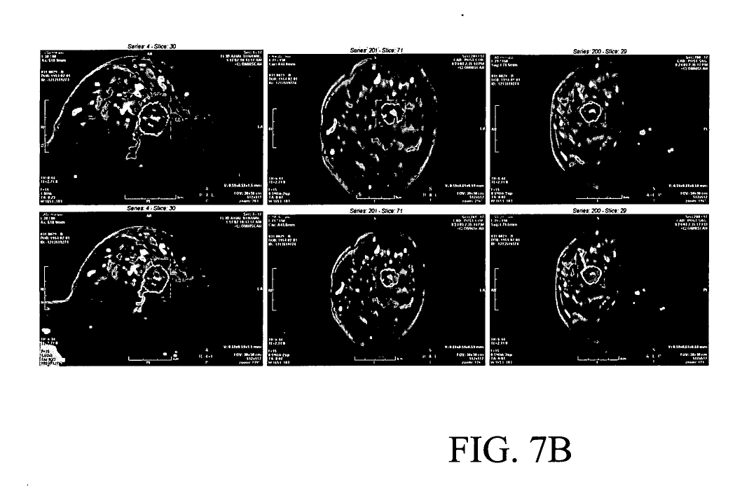

FIG. 6 is a series of images illustrating a lesion at different orientations and resolutions and includes images with and without image labels. FIGS. 7a and b illustrate a series of images with angiomap overlays at different orientations and at different zoom levels. An angiomap provides an indication of vascularization that may be associated with a lesion.

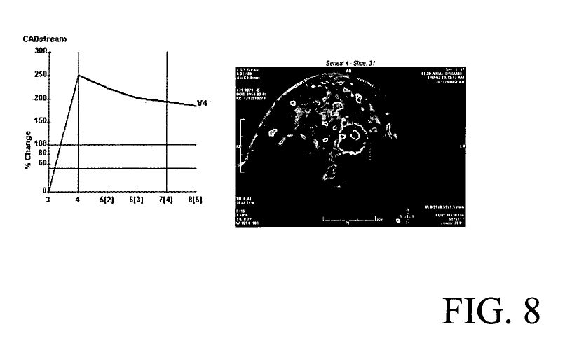

FIG. 8 illustrates an image of a lesion and an associated enhancement characteristic curve for that lesion. As will be discussed in greater detail below, it is known that breast tumors rapidly absorb contrast-enhancing agent and rapidly wash out the contrast-enhancing agent over a relatively short period of time. This curve, sometimes referred to as a washout characteristic curve, may be used to differentiate between harmless masses and cancerous tumors. Washout characteristic curves may be readily used by the radiologist for diagnostic purposes, but may have diminished value for a surgeon.

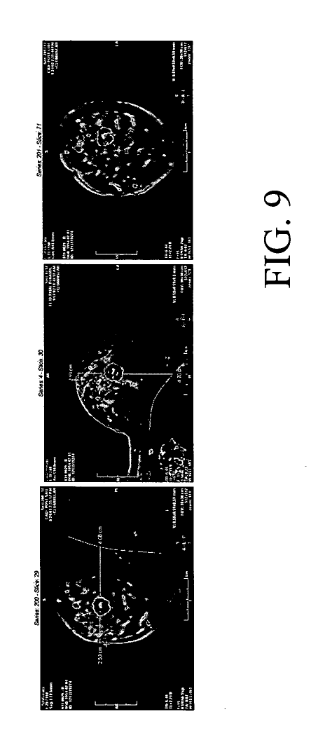

FIG. 9 illustrates elements of a lesion with respect to identified landmarks. These various images and data are stored in the data storage 106 and selectively extracted to automatically form the desired customized report.

Identifying Lesions and Landmarks

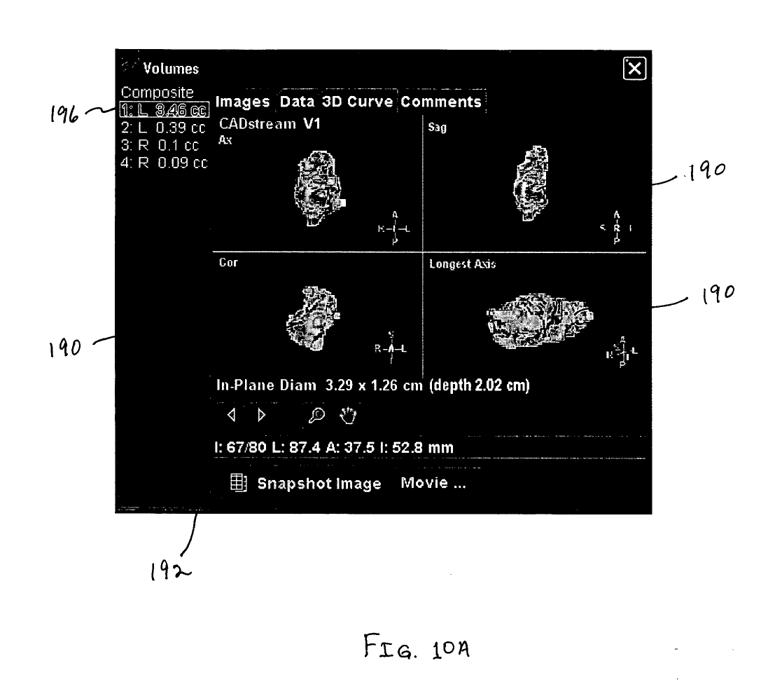

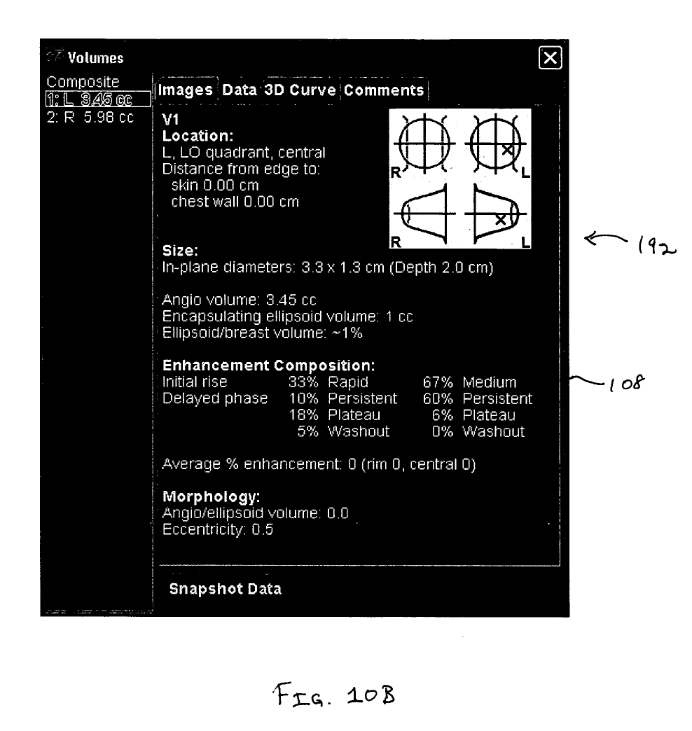

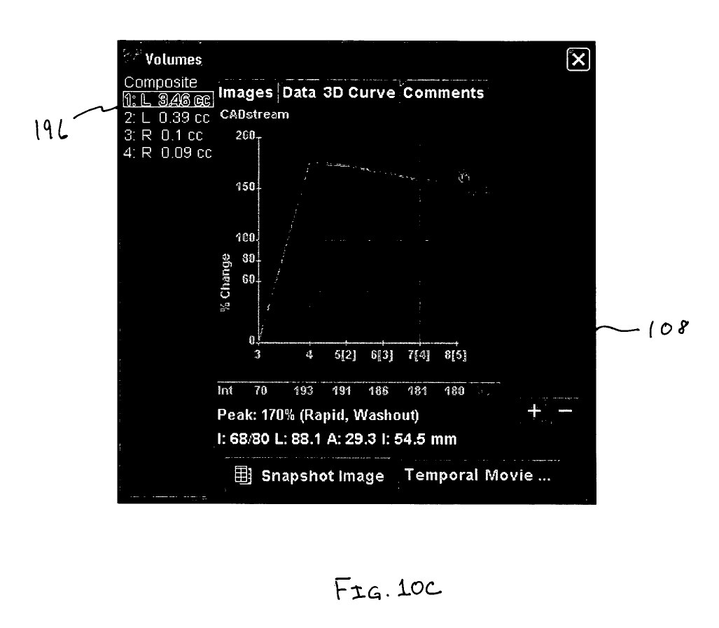

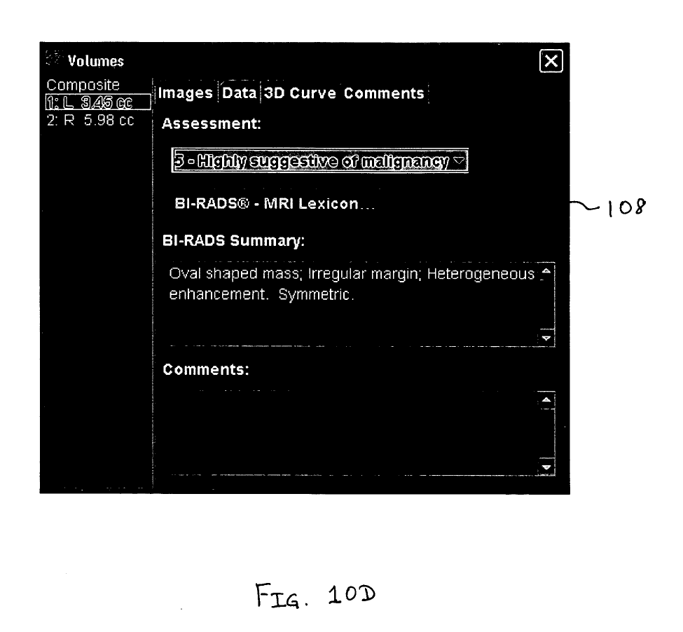

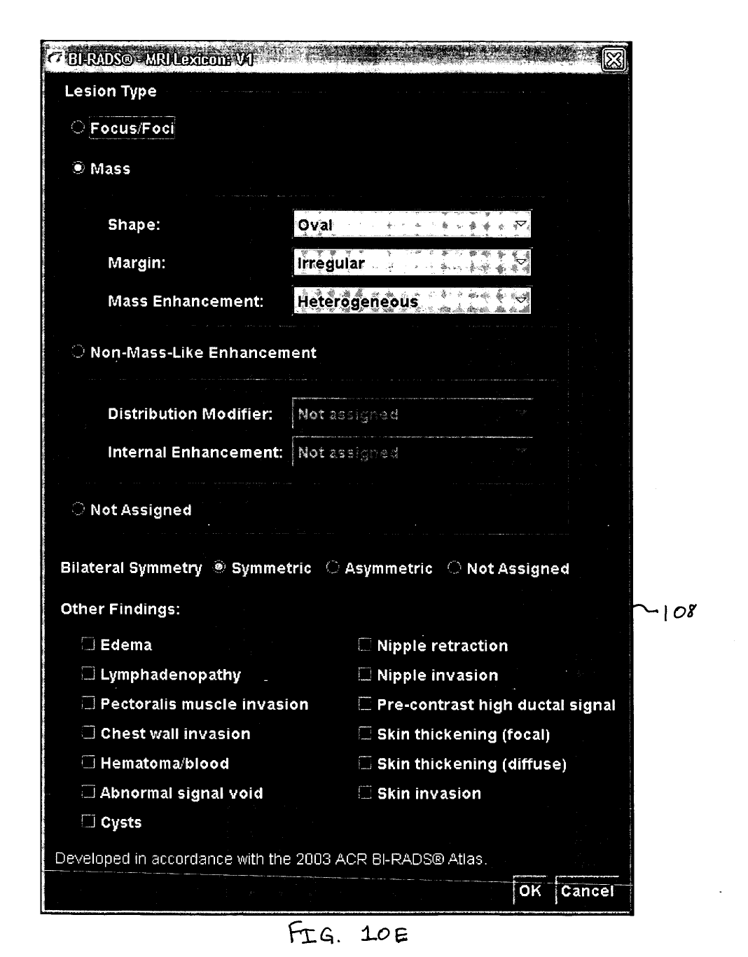

One example application of the system for the creation of reports is illustrated in FIGS. 10-14. A caregiver, typically a radiologist, creates the pre-treatment report at step 140 by analyzing the imaged data and identifying all VOIs. The system 100 advantageously allows individuals to tailor reports for their specific needs. As illustrated in FIG. 1, a radiologist may want a diagnostic report for analysis purposes, while a surgeon can specify different images and views in the form of a surgical planning report. FIGS. 10A-10E illustrate screen displays that a radiologist may use for diagnostic purposes. FIG. 10A illustrates an image of a VOI 190 shown on the display 108 (see FIG. 2) and the associated measurement data generated by the CAD processor (not shown). Other VOIs may be viewed by selecting from a list 196. As illustrated in FIG. 10A, the VOI 190 is shown in different views, such as an axial view, sagittal view, and coronal view. In addition, the CAD processor can analyze the VOI 190 and determine its longest axis to better illustrate the extent of the lesion.

FIG. 10A illustrates one example of an image that may be desired in a pre-treatment report or a surgical planning report. In addition to showing the VOI 190 on the display 108, the user may select a “Data” tab on the display 108 to display measurement data 192 associated with a selected lesion. The measurement may be automatically performed or may be manually determined in conjunction with the cursor control 110. The measurement data includes the three-dimensional diameter of the VOI 190 as well as the length and width of the particular image slice being displayed on the display 108. The CAD processor also calculates the angio volume of the VOI 190. The angio volume indicates the portions of the tumor exhibiting angiogenesis. The measurement data also includes information about an encapsulating ellipsoid. This may be most useful to surgeons. Diameter lengths of the ellipsoid is reported, as well as its volume and the percentage of the ellipsoid volume to the total breast volume.

In addition to measurement data, the display 108 provides data relating to a curve peak, which is an indication of the percent enhancement with pre- and post-contrast data. As previously discussed, tumor cells typically exhibit a medium or rapid uptake of contrast agent and percent enhancement measurement is frequently used to indicate potentially cancerous lesions. In addition to rapid uptake of contrast agent, cancerous cells tend to demonstrate a sudden decrease or washout of the contrast agent. Thus, certain cells indicate a rapid uptake followed by a rapid washout of cells. Other cells indicate a rapid uptake but the percent enhancement tends to peak and form a plateau. Still other cells tend to have a rapid uptake of contrast agent within a short period of time and continue to show a persistent or continuous enhancement. Characterizing the initial rise and the delayed phase of the enhancement curve is also important in the BI-RADS classification.

The display 108 includes composition data that divides the cells within the VOI 190 into one of these subcategories. The percent of the data elements in a particular VOI (e.g., the VOI 190) that have medium versus rapid initial rise enhancement is reported. In addition, the composition of the delayed phase (persistent, plateau, or washout) for each of these data elements is also reported. That is, in the example illustrated in FIG. 10B, 33% of the data elements or voxels that make up the VOI 190 exhibit a rapid uptake of the contrast agent while 67% of the data elements or voxels exhibit medium uptake characteristics in the initial rise. Furthermore, the data of FIG. 10B show that the 10% of the data elements in the VOI 190 exhibited rapid uptake with persistent enhancement behavior, 18% of the data elements in the VOI exhibited rapid uptake with plateau behavior (i.e., there is a rapid uptake of the contrast agent causing an enhancement of the imaging followed by a plateau in which the percent enhancement remains substantially constant). Finally, the data displayed in FIG. 10B illustrates that 5% of the data elements in the VOI 190 exhibited rapid uptake with washout characteristic behavior. Similarly, 60% exhibit medium uptake with persistent behavior, 6% exhibit medium uptake with plateau behavior and 0% exhibit medium uptake with washout behavior. The physician can use this composition data to determine whether a particular VOI (e.g., the VOI 190) is a cancerous lesion or some noncancerous mass. Thus, the image of FIG. 10, in the associated data, may be specified by the radiologist as part of a diagnostic report. The radiologist uses the diagnostic report to evaluate lesions, such as the VOI 190 to determine the likelihood of the VOI 190 being cancerous. The surgeon may use the enhancement composition to monitor treatment response.

Data related to the enhancement characteristics for the selected lesion is illustrated in FIG. 10B. This includes, but is not limited to, other calculations and measurements of the VOI, such as the distribution of the enhancement in the rim versus the center of the VOI. Size and morphological measurements are also included, such as the volume of the lesion, the ratio of its volume to the entire breast volume, the eccentricity or roundness of the lesion, and the ratio of the lesion volume to the encapsulating ellipsoid volume. The enhancement curve itself may be viewed by selecting a “3D Curve” tab on the display 108, resulting in the display of a curve, such as illustrated in FIG. 10C. This curve represents the area of the VOI that has the highest uptake within the 3D volume.

As the physician reviews the case, the physician chooses to keep a VOI in the list to report. Thus, the system 100 provides a convenient technique for listing all VOIs that are suspicious or identified as tumors. The physician may also classify the lesion according to the ACR BI-RADS classification for Breast MR, or enter other comments by selecting a “Comments” tab on the display 108, resulting in the display of FIG. 10D. The physician may select the ACR BI-RADS classification using a drop-down menu. The physician may also select a “BI-RADS MRI lexicon button, illustrated in FIG. 10D to permit the display of additional data related to the VOI using BI-RADS terminology, as illustrated in FIG. 10E. Selections in FIG. 10E may be automatically completed by the CAD processor or manually by the physician. Alternatively, the physician may alter the automatically completed data elements in FIG. 10E.

All the data from the various VOIs in images, measurements, classifications, and other data are stored in the data storage 106 and may be used in a preparation of several different report types, such as a diagnostic report, a pre-treatment report, a surgical planning report or the like.

FIGS. 14A-14G provide an example diagnostic report. Automatic generation of a report includes evaluating and selecting image data for display, associated location data, such as distance from landmarks, calculated measurement data, associated medical test data, and the like.

One skilled in the art will appreciate that medical image data, such as MRI data, typically includes a large number of images. For example, breast imaging often involves the administration of a contrast agent. In the moments following the administration of the contrast agent, a series of images, perhaps 100 or more, are obtained. In addition, images may be obtained from different orientations, such as a series of sagital images, a series of coronal images, and the like. Furthermore, those skilled in the art will appreciate that a typical MRI series contains a plurality of “slices” representing different image planes within the imaged portion of the patient anatomy. The system 100 automatically evaluates a large number of available images to select one or more images that best depict the VOI. Thus, the system advantageously analyzes a large number of images and selects the most appropriate images for inclusion in the report. This is a considerable savings in time from the conventional technique that requires the radiologist to manually evaluate all images to determine which few images to include in the report.

To illustrate the concept of automatic report generation, consider the image of FIG. 14D, which is a one page report on a selected lesion. FIG. 14D includes 6 images selected from a superset of medical images for the particular patient. The report may include image identification information that permits the retrieval of original images or the evaluation of related images. For example, it may be desirable for a surgeon to evaluate multiple slices of a particular VOI to better understand the shape and position of a particular VOI.

The system 100 analyzes different slices to determine the slice with the largest cross-sectional area. The image having the largest cross-sectional area may be included as a selected image. In addition, the system 100 may evaluate a series of slices to determine a centroid for the selected VOI. In addition, the system 100 may evaluate multiple images to determine a volume surrounding the VOI. As previously noted, the surrounding volume may be characterized as an ellipsoid to assist the surgeon in surgical planning for possible removal of the VOI.

In one embodiment, the system 100 may select images based on the location of the VOI. This permits the selection of images that best illustrate the location of the VOI. As illustrated in FIG. 14D, the location may also be illustrated on a wire frame model.

In another embodiment, the images may be selected for inclusion in a report on the basis of size. That is, the system 100 may evaluate images to select one or more images that best illustrate the size of the VOI. The system 100 may also include one or more images based on both location and size.

As illustrated in FIG. 14D, size and location information is calculated and displayed for the selected VOI. The system 100 automatically analyzes multiple images to determine data, such as the longest ellipsoid diameter or in-plane diameters.

FIG. 14A provides a summary of findings, including thumbnail MRI images, classification, and location information for each VOI. FIGS. 14B and 14C provide more detailed images. FIG. 14B provides a full maximum intensity projection (MIP) image while FIG. 14C provides a rendering of the breast with the identified volumes.

FIGS. 14D-14F provide detailed information for each individual VOI. In the example illustrated in FIGS. 14A-14G, three VOIs are illustrated. Those skilled in the art will recognize that an actual report may contain more or less VOIs.

FIG. 14G provides supplemental images identified by the physician to include in the report. These may be images from different scanned sequences that highlight areas of interest to the physician, such as the lymph nodes.

As described above, FIGS. 14A-14G may be automatically generated as a diagnostic report illustrating the findings of the physician. The system 100 advantageously allows the physician to customize reports to suit the particular needs of the individual. In one aspect, the system 100 selects images and data to automatically generate a report, such as the diagnostic report illustrated in FIGS. 14A-14G.

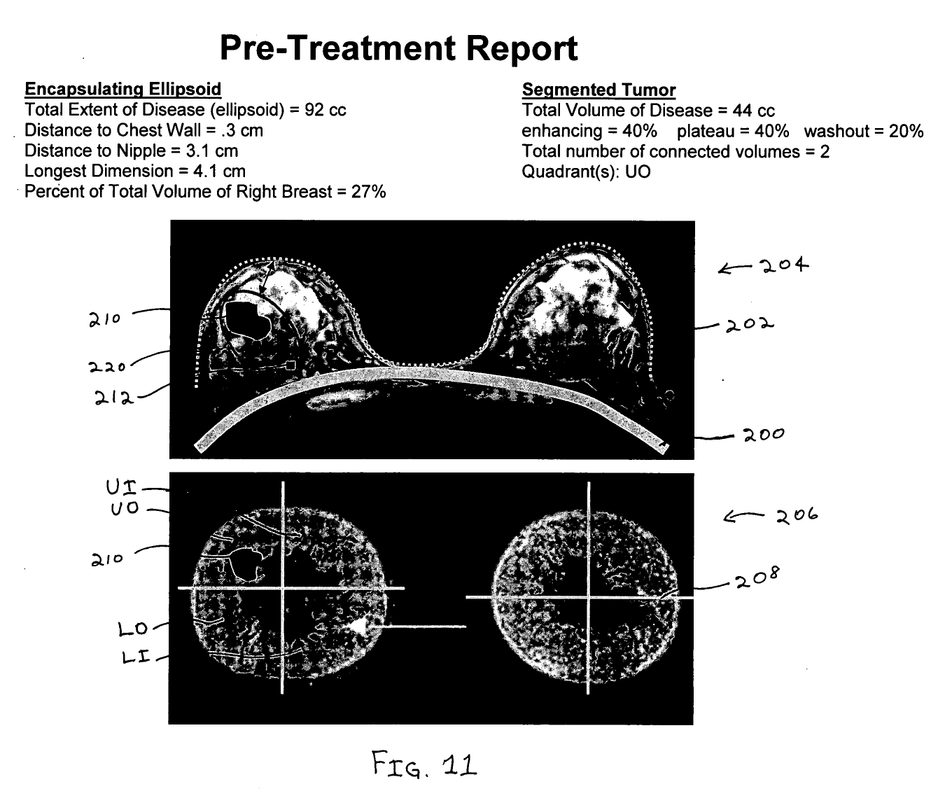

In another aspect, the system 100 can be used as a surgical planning tool. The surgeon may view a selected subset of images that form the diagnostic report to confirm the diagnosis of cancerous lesions. However, the surgeon may also specify selection characteristics that would form the basis of a surgical planning report for use by that particular physician. In this aspect, the system 100 utilizes the selection characteristics for that physician for a surgical planning report, which are stored in the report selector data module 116 (see FIG. 2). The report data filter module 120 applies the selection characteristics to the images and data in the data storage 106 and the report generator module 118 generates a surgical planning report that has been customized for the physician to use for surgical planning purposes. An example pre-treatment report is illustrated in FIG. 11, which includes a transverse axial image 204 of the breast and a coronal image 206 of the breasts. As part of the preparation of the diagnostic reports, a chest wall 200 and skin surface 202 are illustrated in the transverse axial image 204 while a crosshair 208 is positioned on the nipple and the coronal image 206. These landmarks are used by the surgeon for surgical preparation. The crosshairs in the coronal image 206 are used to subdivide each breast into quadrants, which are identified as upper, inner and outer quadrants, UI and UO, respectively, and lower inner and outer quadrants, identified as LI and LO, respectively, for each breast.

Also illustrated in FIG. 11 are a large VOI 210 and a small VOI 212. As can readily be seen from the coronal image 206, the VOI 210 is located in the upper outer (UO) quadrant of the breast.

Those skilled in the art will recognize that the VOIs may not be visible in all images. For example, the transverse axial image 204 shows both the VOI 210 and the VOI 212 while the coronal image 206 shows only the VOI 210. The inability to view the VOI 212 in the image 206 may be due to the fact that the VOI is in a different image plane and thus not visible in the particular image plane selected as the image 206. The VOI 212 may also be hidden behind the VOI 210 and thus not visible in the coronal image 206. As can be readily seen in FIG. 11, the use of anatomical markers, such as the cross-hair 208 and the chest wall 200, aid the physician in locating the VOIs 210 and 212. FIG. 11 also illustrates an ellipsoid 220 generated by the CAD processor.

An ellipsoid, such as the ellipsoid 220, is illustrated as encapsulating the tumors (i.e., the VOI 210 and the VOI 212 of FIG. 11). The use of ellipsoid shaped volumes is selected to correspond with the shape of tissue volume generally removed by surgeons when excising a lesion. However, those skilled in the art will appreciate that other shapes may be used, that the ellipsoid is only one of many different modeling volumes. By encapsulating the VOI 210 and the VOI 212 within the ellipsoid 220, the surgeon can determine the volume of breast tissue that must be removed in order to remove the lesions.

The pre-treatment report also includes measurement data related to the VOIs 210 and 212 as well as measurement data related to the encapsulating ellipsoid 220. Data related to the VOIs 210 and 212 include, by way of example, the number of VOIs identified by the CAD processor was well as the total volume of the VOIs. Location data within a particular quadrant is also indicated. The data related to the segmented tumor (i.e., the VOI 210 and the VOI 212) also includes the total volume of the VOIs. In the example illustrated in FIG. 11, the number of connected volumes (i.e., VOIs within the ellipsoid 220) is two and the total volume of the VOIs is 44 cubic centimeters (cc).

In addition, the pre-treatment report may include contrast imaging data. As previously discussed, contrast imaging may be used to differentiate between normal cells and cancer cells. The pre-treatment report illustrated in FIG. 11 includes data indicating the characteristic composition of the VOIs is also provided. In the example illustrated in FIG. 11, 40% of the data elements (i.e., voxels) associated with the VOI 210 and the VOI 212 exhibit persistent enhancement characteristics while 40% of the data elements exhibit plateau characteristics. Twenty percent of the elements associated with the VOI 210 and the VOI 212 exhibit washout characteristics.

For surgical planning purposes, the pre-treatment report also includes data relating to the ellipsoid 220 that surrounds the VOIs 210 and 212. In the example illustrated in FIG. 11, the ellipsoid 220 surrounds both the VOI 210 and the VOI 212. Alternatively, the surgeon may determine that separate ellipsoids are warranted. In this situation, the system 100 may generate a separate ellipsoid around each VOI. Such decisions are generally based on the size and location of VOIs with respect to each other. The final decision as to the number of ellipsoids may be left to the discretion of the surgeon.

The data for the ellipsoid 220 may include the total volume of the ellipsoid as well as the percent of the ellipsoid volume compared to the total volume of the breast. The ellipsoid data also includes measurement data indicating, by way of example, the distance to the chest wall, the distance to the nipple, and the longest dimension of the ellipsoid 220. In the example of FIG. 11, the ellipsoid 220 includes a volume of 95 ccs, which is 27% of the volume of the right breast. The ellipsoid data also indicates that the distance from the ellipsoid 220 to the chest wall is approximately 0.3 centimeters (cm) while the distance to the nipple is approximately 3.1 cm. The longest dimension of the ellipsoid 220 is 4.1 cm.

However, in another aspect, the system 100 may generate custom reports used not only for surgical planning, but for treatment monitoring. For example, the surgeon may use the pre-treatment report of FIG. 11 to plan breast conserving surgery. The surgery is performed and post-therapy scanning and CAD processing occurs. That is, the system 100 may utilize the CAD processor to monitor lesions or VOIs (e.g., the VOI 190 of FIG. 10) following surgery.

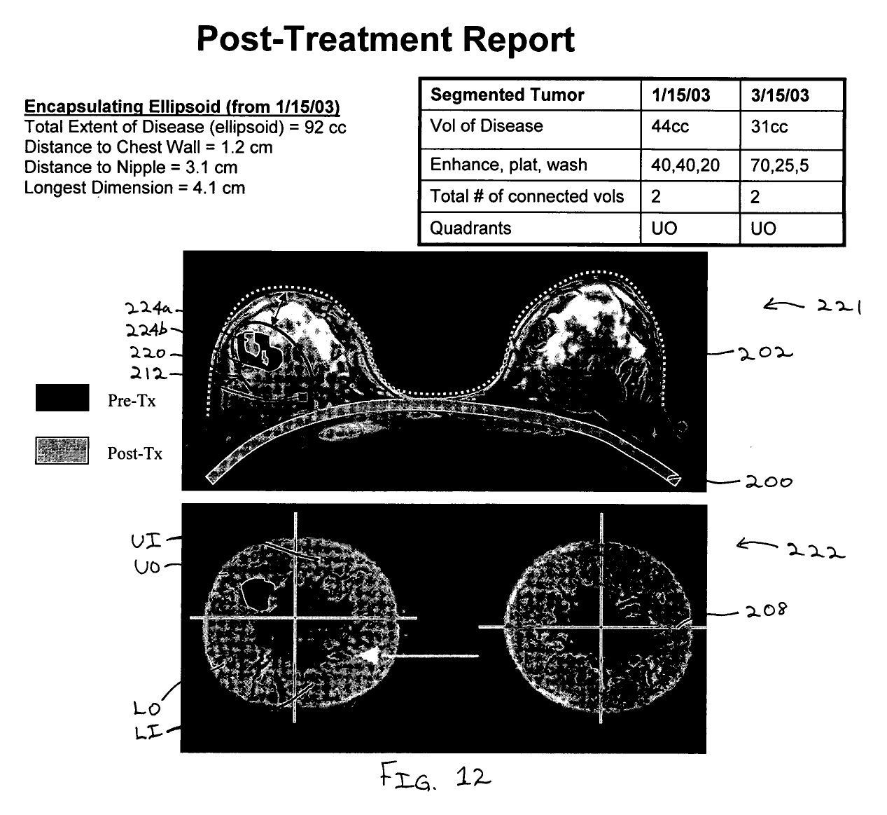

Following surgery, the system 100 creates a post-treatment report. An example of a post-treatment report is illustrated in FIG. 12. Details of post-treatment reports are provided below. The surgeon uses the report to assess surgery or plan additional surgery. Those skilled in the art will appreciate the various stages of this process may be repeated as warranted.

In a yet another aspect, the system 100 can generate reports, such as a response to therapy report, illustrated in FIG. 1. Such a report is useful for an assessment of presurgical treatment, such as the administration of Neo-Adjuvant chemotherapy. It is well-known that chemotherapy and/or radiation therapy may be used to reduce the size of tumors prior to surgery. As previously discussed, the surgeon may elect to perform surgery based solely on the pre-treatment report. The surgery may be in the form of a mastectomy or breast conserving surgery, such as a lumpectomy. Alternatively, the surgeon may elect chemotherapy or other pre-surgical treatment in an effort to reduce the size of the tumor and, in turn, the volume of tissue that will be removed during the surgical procedure. Following one or more cycles of pre-surgical therapy (e.g., chemotherapy), the system 100 creates a post-treatment report.

The advantage of customized report viewing with the system 100 is that it can readily monitor progress of pre-operative treatment, such as a reduction in tumor size, and thereby give the surgeon the greatest amount of useful information regarding the size and location of tumors.

The surgeon can use the pre-treatment report (e.g., the pre-treatment report of FIG. 11) as the baseline for such treatment. The chemotherapy is administered to the patient and a post-therapy scan and CAD processing is performed. The CAD processor (not shown) is used in the manner described to monitor the detected tumors.

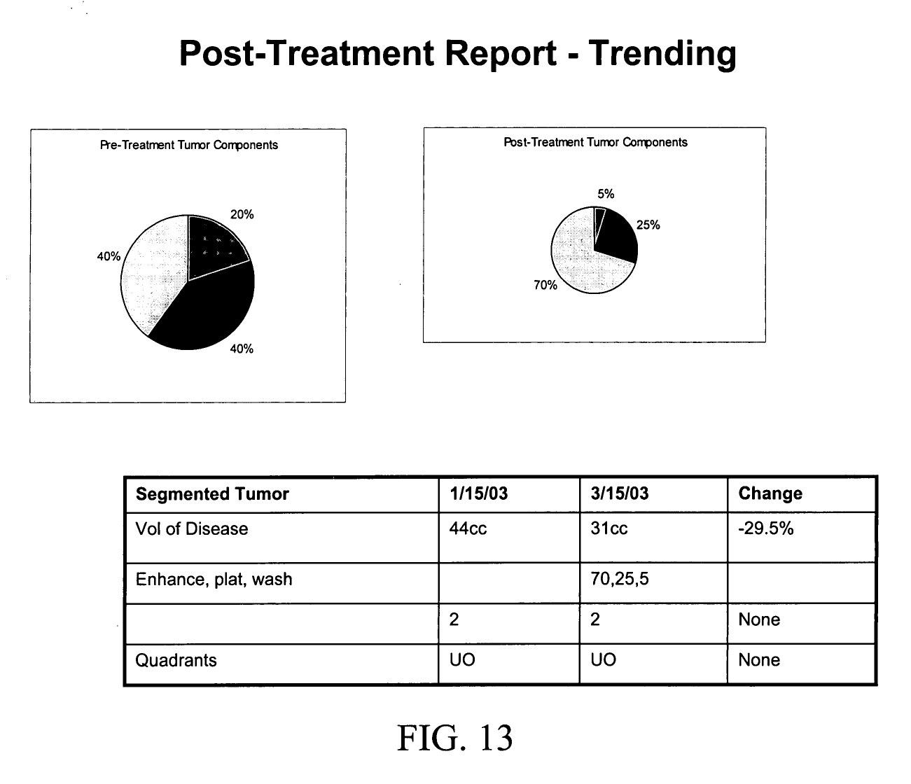

The system 100 is used to create a post-treatment report using predetermined and customized selection criteria stored in the report selector data module 116 (see FIG. 2). FIG. 12 illustrates an example of a post-treatment report. Additional data, such as post-treatment trending data, illustrated in FIG. 13, may also be generated for use by the surgeon. Each of these reports may be custom selected by the surgeon and selects only the images and associated data that is required by the surgeon to perform the assessment.

Because the size, shape and position of the breast may have changed from one imaging session to another, registration, or alignment, of the pre- and post-treatment volumes is required. For the sake of simplicity in the registration process, the breast may be modeled as a rigid body.

The registration process also includes the registration of the cross-hair 208 as well as alignment of the chest wall 200 and the skin surface 202 in the various images. In one embodiment, the registration process may be automatically performed by the system 100. In an alternative embodiment, the coronal and transverse three dimensional views may be registered or aligned by the user using the cursor control 110 (see FIG. 2) to manipulate or align the images on the display 108.

Upon completion of the registration process, the original VOIs may be shown on the display from the pre-treatment report. In the example illustrated in FIG. 12, the VOI 210 and the VOI 212 are illustrated in images 221 and 222. In the example pre-treatment and post-treatment reports of FIGS. 11 and 12, respectively, it should be noted that the image 221 in the post-treatment report corresponds to the image 204 in the pre-treatment report (see FIG. 11) while the image 222 in the post-treatment report corresponds to the image 206 in the pre-treatment report.

In addition to showing the pre-treatment VOIs (i.e., the VOI 210 and the VOI 212), the post-treatment report illustrates VOIs following treatment (i.e., post-treatment VOIs). In the example of FIG. 12, the original VOI 210 has been reduced in size and fragmented into two separate VOIs, illustrated in the transverse image 221 in FIG. 12 as a VOI 224a and a VOI 224b. The image 221 also indicates that the adjuvant chemotherapy has eliminated the VOI 212. In the coronal image 222, the post-treatment VOIs overlap, resulting in an image that appears to show a single VOI 224a, b. Alternatively, the VOI 224b may be in a different image slice and thus not visible in the coronal image 222. The advantage of two views, such as the transverse image 220 and the coronal image 222 is that the surgeon may see multiple VOIs that overlap in one image or another.

The surgeon uses to post-treatment report to assess the Neo-Adjuvant chemotherapy treatment. Based on the custom report, the surgeon may elect to return the patient for additional chemotherapy treatment. Multiple cycles of chemotherapy and post-treatment scanning and reporting may be performed as deemed necessary by the surgeon.

Following one or more cycles of chemotherapy and post-therapy scanning and reporting, the surgeon may perform a mastectomy, if warranted, or may plan breast conserving surgery. In either event, the Custom report viewer of the system 100 can provide the physician with precisely the custom reports necessary to plan the surgery and, post-operatively, to ensure that all suspect tissue has been removed. As is known in the art, positive margins, or reoccurrence in cancer, is not uncommon in breast cancer surgery. However, with the custom reporting provided by the system 100, the surgeon has an opportunity to plan the surgical procedure so as to minimize the chances of a positive margin. In addition, further customized reports can be used to readily identify positive margins if they should occur.

The images illustrated in the present application are black and white or grayscale images. However, those skilled in the art will appreciate that the display 108 (see FIG. 2) is typically a color display. Accordingly, the system 100 takes advantage of color display capability by identifying different VOIs in different colors. For example, the pre-treatment VOIs 210 and 212 may be shown in one color in the pre-treatment report of Figure and the post-treatment report of FIG. 12. The post-treatment VOIs 224a and 224b may be shown in the post-treatment report of FIG. 12 in a different color so as to indicate any change in the VOIs with greater clarity. The specific colors used for pre-treatment and post-treatment display of VOIs may be based on known factors, such as ease of visibility, good contrast between colors, and the like. The system 100 is not limited by any specific color selection. In an alternative embodiment, different graphic patterns may also be used to help differentiate between pre-treatment VOIs and post-treatment VOIs.

The post-treatment report illustrated in FIG. 12 also includes data regarding the segmented tumor and the encapsulating ellipsoid. In an exemplary embodiment, the post-treatment report includes tumor data from the pre-treatment report as well as post-treatment display of the same data. In the example of FIG. 12, the post-treatment report includes the number of identified VOIs, the location of the VOIs and the volume of the tumors based on the pre-treatment report and the post-treatment report. In addition, the percent of VOI tissue exhibiting persistent enhancement, plateau and washout characteristics, as described above, are shown on the report for both pre-treatment and post-treatment. Using the measured data provided in the post-treatment report combined with the images 221 and 222 in the post-treatment report, the surgeon can evaluate the success of the adjuvant chemotherapy. The post-treatment report illustrated in FIG. 12 also provides the measurement data of the original ellipsoid 220.

The post-treatment report can also include trending data to provide the physician with further information regarding the progress of adjuvant chemotherapy. An example of trending data provided in the post-treatment report is illustrated in FIG. 13. The data in the example of FIG. 13 includes measurement data, such as that described above with respect to FIG. 12 as well as calculations regarding changes in data. For example, the volume of the disease (i.e., the tumor) in the pre-treatment report was 44 cc while the volume of the tumor in the post-treatment report is 31 cc. This indicates a 29.5% decrease in volume. The trending report in FIG. 13 can also show the change in the number of connected volumes (i.e., VOIs). An increase in the number of connected volumes may be the result of the cancer mass or volume breaking into multiple smaller pieces. The trending data can also be used to indicate, by way of example, lack of change due to the adjuvant chemotherapy treatment. In such case, the tumor size may be the same or larger.

The post-treatment report of FIG. 13 also includes graphical data to indicate the relative change of tumor components. As previously discussed, the tumor components may be classified by their ability to take up and washout image contrast agents. In the example illustrated in FIG. 13, the percentage of the tumor comprising cells exhibiting washout characteristics dropped from 20% to 5%. At the same time, the percentage of cells exhibiting plateau characteristics dropped from 40% to 25% while the percentage of cells exhibiting persistent enhancement characteristics rose from 40% to 70%. Changes in the composition of the tumor may serve as an indication of the effectiveness of the adjuvant chemotherapy. The characteristic data is also shown in the form of a pie chart in FIG. 13. In an alternative embodiment, the overall size of the pie chart may be altered to reflect the change in the overall tumor volume. Thus, the post-treatment pie chart is somewhat smaller to indicate the 29.5% reduction in the volume.

The physician advantageously uses the system 100 to generate custom reports that provide the physician with precisely the images and data by which the physician can judge the efficacy of adjuvant chemotherapy treatment pre-operatively. The physician may further use the information generated by the system for surgical planning purposes. The location, volume and shape of VOIs permit the surgeon to extract the tumor and a sufficient volume of surrounding tissue so as to minimize the occurrence of positive margins.

The system 100 may also be used to generate custom reports that permit the physician to monitor post-operatively for positive margins. If additional surgery is required, the system 100 can generate the necessary custom reports for surgical planning and monitoring. Thus, the system provides great advantage to the physician pre- and post-operatively for monitoring purposes, for surgical planning purposes, and for analyzing the results of pre-operative therapy. Post-operatively, the system 100 can be used to detect positive margins or the reoccurrence of tumors in another region. The CAD system thereby increases the efficiency of the radiologist interpreting the scan, and the efficiency of the surgeon in managing cancer treatment whether through therapeutic treatment, surgery, or both.

Custom View Setup

The report shall include many images per study. Some of the images represent the entire study, such as a composite view showing all of the identified lesions. For each identified lesion, there are also many images that provide different views and measurements specific to that lesion. The custom view configuration list will identify the global or composite images of interest, as well as the images to show per lesion. Thus, the number of images viewed per study may vary depending on the number of lesions identified for that study.

The physician viewing the report will have a mechanism to choose the desired images to review. Some default configuration lists will be provided for each type of report we have identified: diagnostic, staging, surgical planning, response to therapy. Other default configuration lists may correspond to selections by committees, such as ACR BI-RADS for breast imaging. The physician can start with one of the defaults provided, or create their own from scratch. The list of possible images will be presented in a logical fashion, grouped together by their characteristics. The physician then chooses which images to include in the configuration list. It may be as simple as just clicking on the desired images to select them. Once the configuration list is defined, it is stored in the report selector data module 116 (see FIG. 2) and can be applied to any report of that type, and only the images chosen will be shown when that report type is subsequently selected by the physician. The physician may use the same selection criteria for all report types or custom select selection criteria for each type of report automatically generated by the system 100.

Design Considerations

Below are some of the design considerations to consider when implementing the system 100.

1. Configuration lists will have a study type associated with them. For example, you may have a configuration list for breast MR studies, brain studies, or angiography studies. The report shall include different sets of images depending on the study type.

2. To ensure forward and backward compatibility, image identification “tags” can be assigned for the various image types that will remain consistent between software release versions. Later versions may add image types, but the configuration list still works on all versions.

3. The image identification tags will also identify whether the image is a global image or a per-lesion image. This allows the same image types to be shown for all identified lesions.

4. The image identification tags will also identify whether the image or measurements are per-study. This allows for monitoring results across multiple studies.

5. The custom viewer program should be designed to run on any platform.

6. If the configuration list is from an older version than the report, and the new version report includes some new images, the physician will be prompted to indicate there are more new views to choose from.

The flexible system architecture allows efficient integration into hospital computer systems and hospital workflow. Improvements in efficiency and ease in integration into existing medical systems provides operational and economic advantages as well as increased technological capabilities.

The images shown herein are actual MRI images of breast tissue with volumetric modeling to illustrate the location and size of tumors. In an alternative embodiment, the system 100 may use wire-frame modeling techniques, well known in the art of three-dimensional graphics processing, to illustrate the outline of the breast and landmarks, such as the nipple, chest wall, and skin surface. The use of wire frame modeling eliminates the visual artifact that may be associated with the MRI image data and allows a clear view of the VOI with respect to the wire-frame model.

The foregoing described embodiments depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations.

1. A method for automatic medical report generation, comprising:
performing medical imaging test on a patient to thereby generate medical image data;
identifying anatomical landmarks in the medical image data;
identifying lesions in the medical image data; and
automatically generating a report indicating the identified lesions.

2. The method of claim 1 wherein the report includes image data of selected ones of the identified lesions.

3. The method of claim 1 wherein the medical image data includes a plurality of images of the identified lesions and automatically generating the report comprises evaluating the plurality of images of the identified lesions and selecting at least one of the plurality of images of identified lesions on the basis of lesion location within the patient, the report including the selected ones of the plurality of images of identified lesions.

4. The method of claim 1 wherein the medical image data includes a plurality of images of the identified lesions and automatically generating the report comprises evaluating the plurality of images of the identified lesions and selecting at least one of the plurality of images of identified lesions on the basis of lesion size, the report including the selected ones of the plurality of images of identified lesions.

5. The method of claim 4 wherein the lesion size is determined by calculating a volume of interest (VOI) surrounding the identified lesion.

6. The method of claim 5 wherein the VOI is a substantially ellipsoid volume surrounding the identified lesion.

7. The method of claim 1 wherein the report includes at least one additional data element selected from a group of data elements comprising location data, distance from a landmark data, size data, volume data, enhancement composition data, and morphological indicators data.

8. The method of claim 1 wherein the report includes data conforming to report standards established by ACR BI-RADS.

9. The method of claim 8 wherein the report includes at least one additional data element selected from a group of data elements comprising classification data, location data, distance from a landmark data, size data, volume data, enhancement composition data, characterization data, shape data, boundary data, and comment data.

10. The method of claim 1 wherein the report comprise a graph representing contrast agent uptake and washout characteristics for the area of the identified lesion with the highest uptake.

11. The method of claim 1 wherein identifying lesions comprises manual identification of lesions based at least in part on the medical image data.

12. The method of claim 1 wherein identifying lesions comprises automatic identification of lesions by a computer-aided detection (CAD) processor based at least in part on the medical image data.

13. A method for automatic medical report generation, comprising:
performing medical imaging test on a patient to thereby generate medical image data;
storing the medical image data;
identifying volumes of interest (VOI) in the stored medical image data;
generating additional data related to the VOIs;
generating a full report containing a superset of medical image data and the additional data; and
generating a customized report containing a portion of the full report.

14. The method of claim 13 wherein the customized report is a user-specified report containing portions of the full report specified by a user.

15. The method of claim 14, further comprising accepting user input to select the portions of the full report for the customized report.

16. The method of claim 15, further comprising saving data related to the user-selected portions of the full report for subsequent use to select portions of additional full reports to thereby generate additional customized reports.

17. The method of claim 13 wherein the customized report uses a predetermined customization specifying portions of the full report.

18. The method of claim 17 wherein the predetermined customization specifying portions of the full report conforms to report standards established by ACR BI-RADS.

19. A computer-readable media comprising computer instructions to cause a computer to automatically generate a medical report of medical testing on a patient, the medical testing including medical image data, by causing the computer to:
identify anatomical landmarks in the medical image data;
identify lesions in the medical image data; and
automatically generate a report indicating the identified lesions.

20. The computer-readable media of claim 19 wherein the report includes image data of selected ones of the identified lesions.

21. The computer-readable media of claim 19 wherein the medical image data includes a plurality of images of the identified lesions and automatically generating the report comprises evaluating the plurality of images of the identified lesions and selecting at least one of the plurality of images of identified lesions on the basis of lesion location within the patient, the report including the selected ones of the plurality of images of identified lesions.

22. The computer-readable media of claim 19 wherein the medical image data includes a plurality of images of the identified lesions and automatically generating the report comprises evaluating the plurality of images of the identified lesions and selecting at least one of the plurality of images of identified lesions on the basis of lesion size, the report including the selected ones of the plurality of images of identified lesions.

23. The computer-readable media of claim 22 wherein the lesion size is determined by calculating a volume surrounding the identified lesion.

24. The computer-readable media of claim 23 wherein the volume is a substantially ellipsoid volume surrounding the identified lesion.

25. The computer-readable media of claim 19 wherein the report includes at least one additional data element selected from a group of data elements comprising location data, distance from a landmark data, size data, volume data, enhancement composition data, and morphological indicators data.

26. The computer-readable media of claim 19 wherein the report includes data conforming to report standards established by ACR BI-RADS.

27. The computer-readable media of claim 26 wherein the report includes at least one additional data element selected from a group of data elements comprising classification data, location data, distance from a landmark data, size data, volume data, enhancement composition data, characterization data, shape data, boundary data, and comment data.

28. The computer-readable media of claim 19 wherein the report comprise a graph representing contrast agent uptake and washout characteristics for the area of the identified lesion with the highest uptake.

29. The computer-readable media of claim 19 wherein identifying lesions comprises manual identification of lesions based at least in part on the medical image data and using a computer input device to indicate a lesion.

30. The computer-readable media of claim 19 wherein identifying lesions comprises automatic identification of lesions by a computer-aided detection (CAD) processor based at least in part on the medical image data.

31. A computer-readable media comprising computer instructions to cause a computer to automatically generate a medical report of medical testing on a patient, the medical testing including medical image data, by causing the computer to:
store the medical image data;
identify volumes of interest (VOI) in the stored medical image data;
generate additional data related to the VOIs;
generate a full report containing a superset of medical image data and the additional data; and
generate a customized report containing a portion of the full report.

32. The computer-readable media of claim 31 wherein the customized report is a user-specified report containing portions of the full report specified by a user.

33. The computer-readable media of claim 32, further comprising computer instructions to cause the computer to accept user input to select the portions of the full report for the customized report.

34. The computer-readable media of claim 33, further comprising computer instructions to cause the computer to save data related to the user-selected portions of the full report for subsequent use to select portions of additional full reports to thereby generate additional customized reports.

35. The computer-readable media of claim 31 wherein the customized report uses a predetermined customization specifying portions of the full report.

36. The computer-readable media of claim 35 wherein the predetermined customization specifying portions of the full report conforms to report standards established by ACR BI-RADS.

37. A system to automatically generate a medical report of medical testing on a patient, the medical testing including medical image data, comprising:
a data storage structure to store the medical image data;
a processor configured to:
access the data storage structure;
identify anatomical landmarks in the medical image data;
identify lesions in the medical image data; and
automatically generate a report indicating the identified lesions.

38. The system of claim 37 wherein the report includes image data of selected ones of the identified lesions.

39. The system of claim 37 wherein the medical image data includes a plurality of images of the identified lesions, the processor further configured to evaluate the plurality of images of the identified lesions and select at least one of the plurality of images of identified lesions on the basis of lesion location within the patient, the processor automatically generating the report including the selected ones of the plurality of images of identified lesions.

40. The system of claim 37 wherein the medical image data includes a plurality of images of the identified lesions, the processor further configured to evaluate the plurality of images of the identified lesions and select at least one of the plurality of images of identified lesions on the basis of lesion size, the processor automatically generating the report including the selected ones of the plurality of images of identified lesions.

41. The system of claim 40 wherein the lesion size is determined by calculating a volume surrounding the identified lesion.

42. The system of claim 41 wherein the volume is a substantially ellipsoid volume surrounding the identified lesion.

43. The system of claim 37 wherein the processor is configured to automatically generate the report including at least one additional data element selected from a group of data elements comprising location data, distance from a landmark data, size data, volume data, enhancement composition data, and morphological indicators data.

44. The system of claim 37 wherein the processor is configured to automatically generate the report including data conforming to report standards established by ACR BI-RADS.

45. The system of claim 44 wherein the processor is configured to automatically generate the report including at least one additional data element selected from a group of data elements comprising classification data, location data, distance from a landmark data, size data, volume data, enhancement composition data, characterization data, shape data, boundary data, and comment data.

46. The system of claim 37 wherein the processor is configured to automatically generate the report comprising a graph representing contrast agent uptake and washout characteristics for the area of the identified lesion with the highest uptake.

47. The system of claim 37 wherein identifying lesions comprises manual identification of lesions based at least in part on the medical image data, the system further comprising a computer input device to indicate a lesion.

48. The system of claim 37 wherein the processor is a computer-aided detection (CAD) processor configured to automatically identify lesions based at least in part on the medical image data.

49. The system of claim 37 wherein the medical testing includes additional data related to the image data and the data structure stores a superset of medical image data and the additional data, the system processor further configured to generate a customized report containing a selected portion of the superset of medical image data and the additional data related to the selected portion of the superset of medical image data.

50. The system of claim 49, further comprising an input device operable by a user to specify the selected portion of the superset of medical image data and the additional data related to the selected portion of the superset of medical image data to include in a user-specified customized report.

51. The system of claim 50 wherein the processor is further configured to save data related to the user-specified customized report in the data storage structure for subsequent use to select portions of additional supersets of medical image data and the additional data to thereby automatically generate additional customized reports.

52. The system of claim 49 wherein the customized report uses a predetermined customization to specify the selected portions of the superset of medical image data and the additional data related to the selected portion of the superset of medical image data.

53. The system of claim 52 wherein the predetermined customization specifying portions of superset of medical image data and the additional data related to the selected portion of the superset of medical image data conforms to report standards established by ACR BI-RADS.
